(19) United States
(12) Patent Application Publication
(10) Pub. No.: US 2025/0295651 A1
(43) Pub. Date: Sep. 25, 2025

(54) METHODS OF USING ALTERNATING ELECTRIC FIELDS IN COMBINATION WITH TEMOZOLOMIDE AND A CHECKPOINT INHIBITOR

(71) Applicant: NOVOCURE GMBH, Baar (CH)

(72) Inventors: David D. Tran, Los Angeles, CA (US); Dongjiang Chen, Alhambra, CA (US)

(21) Appl. No.: 19/090,173

(22) Filed: Mar. 25, 2025

Related U.S. Application Data
(60) Provisional application No. 63/569,568, filed on Mar. 25, 2024.

(51) Int. Cl. A61K 31/495; A61K 39/395; A61N 1/36; A61P 35/00

(52) U.S. Cl. CPC A61K 31/495; A61K 39/3955; A61N 1/36002; A61P 35/00

(57) ABSTRACT

Disclosed are methods of treating a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject. Disclosed are methods of increasing survival of a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject.

### CROSS-REFERENCE TO RELATED APPLICATIONS

This application claims the benefit of U.S. Provisional Patent Application No. 63/569,568, filed Mar. 25, 2024, which is incorporated by reference herein in its entirety.

### BACKGROUND

The survival outlook for glioblastoma (GBM), the most prevalent primary CNS cancer in adults, remains bleak. Even with aggressive standard of care including maximal surgical resection, followed by adjuvant chemoradiation and Tumor Treating Fields (TTFields), the median overall survival (OS) is 20.9 months and the 5-year survival rate stands at a mere 13%. For patients presenting with tumors deemed inoperable due to comorbid conditions or localization within eloquent brain regions, prognostic expectations are considerably worse with median OS of less than 12 months. This stark reality emphasizes an imperative need for the development and integration of novel therapeutic modalities that can improve clinical outcomes, particularly for patients harboring substantial tumor burdens that are beyond the scope of surgical excision.

### BRIEF SUMMARY

Disclosed are methods of treating a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject.

Disclosed are methods of increasing survival of a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject.

Additional advantages of the disclosed methods and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1C shows a clinical study design. FIG. 1A) The study schema and objectives testing adjuvant TTFields plus pembrolizumab and TMZ in patients with newly diagnosed GBM and the sample collection plan for correlative analysis. Pembrolizumab was added at cycle 2 of TMZ and TTFields. FIG. 1B) A CONSORT flowchart detailing recruitment and enrollments. FIG. 1C) Baseline characteristics of evaluable patients and treatments in the ITT and wtIDH GBM only populations.

FIGS. 2A-2D show Kaplan-Meier survival curves in the ITT and wtIDH GBM only populations. FIG. 2A) Median PFS from enrollment in the ITT population was 12 months (95% CI, 8.83, 21.1). FIG. 2B) Median OS from enrollment if the ITT population was 24 months (95% CI, 16.1, 29.5). FIG. 2C) Median PFS from enrollment in the wtIDH GBM only population was 10.8 months (95% CI, 7.4, 16.6). FIG. 2D) Median OS from enrollment in the wtIDH GBM only population was 20.5 months (95% CI, 12.5, 25.5).

FIGS. 3A-3D show bulky biopsy-only tumors are associated with higher response and survival. FIG. 3A) A waterfall plot of changes in target lesions as percent of baseline measurements per the iRANO criteria in ITT (left) and wtIDH GBM only (right) populations. FIG. 3B) A Swimmer's plot of individual patient's survival timeline in the maximal resection and biopsy-only cohorts in the wtIDH GBM population. Green diamonds denote time of progression. Red arrow denotes ongoing survival without progression and death at the time of data cut off for analysis. FIG. 3C) For the wtIDH GBM only population, the median PFS from enrollment for the biopsy-only tumor group was 27.2 months compared to 9.6 months for the maximal resection group (hazard ratio [HR] 0.37; 95% CI, 0.16-0.85; log rank P=0.014). FIG. 3D) For the wtIDH GBM only population, the median OS from enrollment for the biopsy-only tumor group was 31.2 months compared to 18.8 months for the maximal resection group (HR 0.4; 95% CI, 0.17-0.92; log rank P=0.023).

FIGS. 4A-4C show the TME of maximal resection and biopsy-only tumors were similar. FIG. 4A) A multivariate Cox fit hazard ratio model of the indicated 20 GO immune pathways with the highest correlation with survival and response in the study cohort. FIG. 4B) Images of multiplex IHC of primary and recurrent tumor samples from Maximal Resection and Biopsy-only patients show colocalization of CX3CR1 and IRF8 in SOX2⁻ cells, indicating their association with the macrophage/microglia population. FIG. 4C) A heatmap of the mean expression of 20 indicated top ranked G.O. pathways with the highest correlation with survival showed no significant difference between maximal resection and biopsy-only tumors.

FIGS. 5A-5C show TCR clonal replacement ratio between C1 and C4 (2 months) of pembrolizumab predicts survival. FIGS. 5A-B) Top panels-Representative 2 non-responder patients with maximal resection who experienced early progressive disease (PD #1 and PD #2) by iRNAO and shortened PFS and OS (FIG. 5A) and 2 responder patients with biopsy-only tumor who achieved complete response (CR #1 and CR #2) by iRANO and had prolonged PFS and OS (FIG. 5B). Bottom panels-Top 20 most expanded TCR clones at each indicated treatment time were tracked across all times, showing that the 2 CR #1 and CR #2 patients exhibited robust TCR clonal replacement by C4 of pembrolizumab. FIG. 5C) Successful TCR clonal replacement from C1 and C4 of pembrolizumab predicts higher response and survival: TCR replacement ratio was detected early in the course of treatment. Analysis was performed using Student T-test. In a multivariate Cox Proportional Hazards Model, high TCR clonal replacement ratio showed high correlation with survival. Concordance statistic was used to assess the performance of the Cox hazards model. The Kaplan Meier plots were calculated using median TCR replacement of low or high replacement ratio. Survival comparison was assessed using log rank test.

FIGS. 6A-6F show peripheral T cells in patients with biopsy-only tumors exhibited higher activation in response to local TTFields and pembrolizumab. FIGS. 6A-D) 3D maps of the activation status of GeneRep/nSCORE-generated global pathway hubs in bulk RNA-seq of enriched peripheral T cells from patients with maximal resection vs biopsy-only tumors in the ITT (FIG. 6A, 6C) and wtIDH GBM only (FIG. 6B, 6D) populations showing that peripheral T cells in patients with biopsy-only tumors exhibited more robust upregulation of the immune regulatory hub 1.1 compared to those in patients with maximal resection, but only after the initiation of the study treatment. Globe size: the number of pathways in a hub; Globe colors: Red—upregulation; Blue—downregulation; Grey—unchanged. Gene names listed after a globe number are master regulators of that hub. FIG. 6E, 6F) Combo box and whisker and dot plots of the mean RNA-seq expression of indicated GO immune regulatory pathways in enriched peripheral T cells at the indicated treatment times from patients with biopsy-only vs maximal resection tumors in the ITT (FIG. 6E) and wtIDH GBM only (FIG. 6F) populations. Data are represented as mean±SEM. Analyses were performed using the paired samples Wilcoxon test in R language.

FIGS. 7A-7H show T cell activation trajectory at the single cell level in representative responders with biopsy-only tumors. FIG. 7A) A 2D UMAP of all T cells at resolution 1 showing 18 major CD4⁺ and CD8⁺ T cell subtypes in the ITT population. FIG. 7B-C) Top panel—Treatment timeline and selective serial brain MRIs of the 2 representative responders with biopsy-only tumors that achieved complete response CR #1 (FIG. 7B) and CR #2 (FIG. 7C). Bottom panels-2D UMAP of T cell clusters with activation status of T cell activation (GO: 0042110) and Adaptive Immune Response (GO: 0002250) pathways, showing progressive immune activation in response to the study treatment with a shift from anergic and naïve T cells to activated T cells and central memory (CM) T cell production. Colored arrows: Red—CM CD8⁺ T cells; Green—Activated CD4⁺ T cells; Black—Effector CD8⁺ T cells; Blue—Effector memory CD8⁺ T cells; and Purple—Anergic CD4⁺ T cells. Red asterisk: CM CD4⁺ T cells. CM T cell production was reversed from first (R1) to second (R2) recurrence in CR #2 patient. FIGS. 7D-E) Violin plots of the mean RNA-seq expression of the T cell activation pathway (GO: 0042110) in all T cells from CR #1 (FIG. 7D) and CR #2 (FIG. 7E). FIGS. 7F-H) Line graphs (top) and associated data tables (bottom) of enumeration of anergic CD4⁺ T cells (FIG. 7F), CM CD8⁺ T cells (FIG. 7G) and CM CD4⁺ T cells (FIG. 7H) as percentage of total T cells (CD3⁺) over the course of study treatment in the same 2 representative responders CR #1 and CR #2 as compared to the same 2 representative non-responders PD #1 and PD #2 patients. Analysis was performed using the paired samples Wilcoxon test in R language.

FIGS. 8A-8D show TCR clonal evolution and activation in representative responders. FIGS. 8A-B) 2D UMAP plots of all CD8⁺ (FIG. 8A) and CD4⁺ (FIG. 8B) TCR clones and the associated mean expression of the T cell activation pathway GO: 0042110 in both CR #1 and CR #2 responders, showing that CD8⁺ TCR clones were selected early in the treatment course and expanded and further activated in subsequent times, while the top expanded CD4+ TCR clones were largely replaced from one treatment time to the next. FIG. 8C) An overlay of the first recurrence R1's and the second recurrence R2's peripheral T cell UMAP graphs showing a leftward shift in R2 from an activated and memory state in R1 to an anergic one in R2. FIG. 8D) 2D maps of the GeneRep/nSCORE-generated immune checkpoint regulatory subnetwork changes in the first recurrence R1's vs the second recurrence R2's peripheral CD8⁺ and CD4⁺ T cells. Shape size: nSCORE importance rank of a gene. Shape colors: Red—upregulated; Blue—downregulated; Grey—unchanged.

FIGS. 9A-9G show TME reprogramming and alternate immune checkpoints contribute to resistance to TTFields and anti-PD-1 immunotherapy. FIG. 9A) A 3D map of the activation status of GeneRep/nSCORE-generated global pathway hubs in bulk RNA-seq expression profiling of 9 paired primary vs recurrent tumor samples in the ITT GBM population. Globe size: the number of pathways in a hub; Globe colors: Red—upregulation; Blue—downregulation; Grey—unchanged. Gene names listed after a globe number are master regulators of that hub. FIG. 9B) GSEA of the representative GO pathways in hubs 1.1 (Hypoxic and Fibrotic Response) and 1.4 (Neural Stemness). NES: normalized enrichment score. FDR: False discovery rate. FIG. 9C) A heatmap of fold change in key markers in the inflammation hub 1.7 in the 9 paired primary vs recurrent GBM tumors in FIG. 9A), showing that hub 1.7 is enriched in pathologic inflammatory response, immune inhibitory and escape gene clusters. FIG. 9D) 2D maps of the GeneRep/nSCORE-generated immune checkpoint regulatory subnetwork changes in the deconvoluted immune (CD45⁺) and non-immune (tumor cells and CD45⁻ stromal cells) TME cells from the 9 paired primary vs recurrent GBM tumors in FIG. 9A), showing the expected compensatory downregulation of the PD-1/PD-L1 axis and the downregulation of IDO1, TIGIT and LAG3, while other alternate immune checkpoints TIM-3/LGALS9, VSIR, PVR and CD276 were regulated. Shape size: nSCORE importance rank of a gene. Shape colors: Red—upregulated; Blue—downregulated; Grey—unchanged. FIG. 9E) GSEA of the representative GO pathways in the inflammation hub 1.7 that have been implicated in regulating immune checkpoints. FIG. 9F) Heatmaps of fold change in expression of major immune checkpoints and MHCI molecules in the 9 paired primary vs recurrent GBM tumors treated in the 2THETOP study confirmed the predicted downregulation of the PD-1/PD-L1 axis, TIGIT and LAG3 and upregulation of the alternate immune checkpoints TIM-3/LGALS9, VSIR, PVR and CD276, which was not observed in a historical dataset of 12 paired primary vs recurrent GBM tumors treated with adjuvant TMZ alone or TMZ plus TTFields (FIG. 9F). FIG. 9G) Representative images of IHC for PD-L1, LGALS9 and VSIR in 2 sets of paired primary vs recurrent tumors in the 2THETOP study. Scale bar=50 μm.

FIG. 10 shows a table with characteristics of patients evaluable for safety.

FIG. 11 shows a table with a toxicity summary that includes all treatment related adverse events.

FIGS. 12A-12C show that bulky biopsy-only tumors are associated with higher response and survival in the ITT population. FIG. 12A) A Swimmer's plot of individual patient's survival timeline in the maximal and biopsy-only cohorts in the ITT population. Green diamonds denote time of progression. Red arrow denotes ongoing survival without progression and death at the time of data cut off for analysis. FIG. 12B) For the ITT population, the median PFS from enrollment for the biopsy-only tumor group was 27.2 months compared to 10.8 months for the maximal resection group (HR 0.58; 95% CI, 0.25-1.34; log rank P=0.231).

FIG. 12C) For the ITT population, the median OS from enrollment for the biopsy-only tumor group was 31.2 months compared to 23.7 months for the maximal resection group (HR 059; 95% CI, 0.25-1.38; log rank P=0.349).

FIGS. 13A-13B show tumor mutational burdens in maximal resection and biopsy-only tumors. Combo box and whisker and dot plots of total functional mutational burden, stop gain SNP, sotp loss SNP and insertions/deletions (IN/DEL) showing minimal differences between maximal resection and biopsy-only tumors in the ITT (FIG. 13A) and wtIDH GBM only (FIG. 13B) populations.

FIG. 14 shows the co-expression of indicated mRNAs were determined by Pearson correlation coefficient. IRF8 expression had high correlation with the macrophage marker CD68. The microglia marker CX3CR1 had high correlation with IFR8 but only moderate correlation with the macrophage marker CD68.

FIGS. 15A-15C show TCRB clonal expansion induced by TTFields but not after the addition of pembrolizumab. (Top, Left) A violin plot of the top 20 TCRB clonal expansion index (inverse of Shannon diversity index), (Top, right) A multivariate Cox Proportional Hazards Model showing concordance of TCRB clonal expansion and survival, and (bottom) a Kaplan Meier plot using median TCRB clonal expansion of low or high expansion ratio in Pre-TTF vs Post-TTF (FIG. 15A), Post-TTF vs C2 (FIG. 15B), and Post-TTF vs C4 (FIG. 15C) in a univariate analysis. TCRB clonal expansion index was analyzed using paired Student T-test. Concordance statistic was used to assess the performance of the Cox HR model. Survival comparison was assessed using log rank test.

FIG. 16 shows TCR clonal replacement between Pre-TTF vs Post-TTF, Pre-TTF vs C2, and Post-TTF vs C2. (Top, left) A violin plot of the top 20TCRB clonal replacement ratio; (Top, right) A multivariate Cox Proportional HR Model showing concordance of TCRB clonal replacement and survival; and (bottom) a Kaplan Meier plot using median TCRB clonal replacement of low or high replacement ratio in a univariate analysis. TCRB clonal replacement ratio was analyzed using paired Student T-test. Concordance statistic was used to assess the performance of the Cox HR model. Survival comparison was assessed using log rank test.

FIGS. 17A-17D show TTFields treatment correlates with immune activation via a T1IFN trajectory in GBM patients. 2D UMAP of all PBMC clusters with activation status of a T1IFN pathway (GO: 0034340) and a non-T1IFN inflammatory pathway (GO: 0002437) pathways in representative CR #1 (FIG. 17a), CR #2 (FIG. 17b), PD #1 (FIG. 17c), and PD #2 (FIG. 17d) patients, showing TTFields-dependent induction of the T1IFN pathway while no significant activation of the non-T1IFN inflammatory pathway following TTFields and TTFields plus pembrolizumab treatment.

FIGS. 18A-18D show T cell activation trajectory at the single cell level in representative non-responders with maximal resection tumors. FIG. 18A, C (top) Treatment timeline and serial brain MRIs of the 2 representative non-responders with maximal resection tumors who experienced early progressive disease PD #1 (18A) and PD #2 (18C). (Bottom) 2D UMAP of T cell clusters with activation status of indicated GO pathways. Arrows: red—CM CD8+ T cells; Green—activated CD4+ T cells; Black—effector CD8+ T cells; Blue—effector memory CD8+ T cells; and purple—Anergic CD4+ T cells. Red asterisk: CM CD4+ T cells. FIG. 18B, D) Violin plots of the mean expression of the indicated GO pathways in all T cells from PD #1 (18B) and PD #2 (18D).

FIGS. 19A-19B show T cell activation in representative responders with biopsy-only tumors. Violin plots of the mean RNA-seq expression of the indicated GO immune pathways in all T cells from CR #1 (FIG. 19A) and CR #2 (FIG. 19B).

FIGS. 20A-20E show key T cell clusters in representative responders with biopsy-only tumors. Line graphs (top) and associated data tables (bottom) of enumeration of naïve CD4+ T cells (FIG. 20A), activated CD4+ T cells (FIG. 20B), effector CD8+ T cells (FIG. 20C), anergic CD8+ T cells (FIG. 20D), and naïve CD8+ T cells (FIG. 20E) as percentage of total T cells (CD3+) over the course of study treatment in the 2 representative responders CR #1 and CR #2 as compared to the 2 representative non-responders PD #1 and PD #2 pateints. Analysis was performed using the paired samples Wilcoxon test in R language.

FIGS. 21A-21B show 3D maps of the activation status changes in GeneRep/nSCORE-generated global pathway hubs in peripheral single CD8+ (FIG. 21A) and CD4+ (FIG. 21B) T cells between the time of the first (R1) vs the second (R2) recurrence of the CR #2 responder. Globe size: the number of pathways in a hub; Globe colors: Red—upregulation; Blue—downregulation; Grey—unchanged. Gene names listed after a globe number are master regulators of that hub.

FIGS. 22A-22B show immune TME of CR #2's primary and recurrent tumors by deconvolution analysis of bulk RNA-seq. FIG. 22A) Deconvoluation of bulk RNAseq of primary, R1 and R2 recurrent tumors in CR #2 patient showing a substantial increases in myeloid signals. FIG. 22B) combo box and whisker and dot plots of mean expression of 10 key immune activation and adaptive immune activation pathways from GO and REactome collections in the TME of R1 vs R2 showing overall high activation status of the adaptive immune signals in R1 vs R2 despite the higher immunosuppressed TME in R2. Analysis was performed using paired student T-test. List of 10 key pathways in immune activation and adaptive immune activation: 1) T cell activation involved in immune response—GO: 002286; 2) regulation of t cell activation-GO: 0050863; 3) activation of immune response—GO: 0002253; 4) immune response-activating signaling pathway-GO: 0002757; 5) reactome immune system-R-HAS-168256; 6) positive regulation of immune system process-GO: 0002684; 7) positive regulation of immune response—GO: 0050778; 8) regulation of immune response—GO: 0050776; 9) T cell activation—GO: 0042110; 10) adaptive immune response—GO: 0002250.

FIGS. 23A-23D show TME reprogramming in recurrent tumors. GSEA of the representative GO pathways in hub 1.1 (Hypoxic and Fibrotic Response) including in migratory regulation (FIG. 23a) and genetic response to hypoxia (FIG. 23b), in hub 1.4 (Neuronal signaling) including various axonic and neurotransmitter signaling (FIG. 23c), and in hub 1.7 (Inflammation) including antigen presentation and inflammatory pathways (FIG. 23d). NES: normalized enrichment score. FDR: False discovery rate.

### DETAILED DESCRIPTION

The disclosed methods and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a checkpoint inhibitor” includes a plurality of such inhibitors, reference to “the checkpoint inhibitor” is a reference to one or more inhibitors and equivalents thereof known to those skilled in the art, and so forth.

The word “or” as used herein means any one member of a particular list and also includes any combination of members of that list.

As used herein a “biopsy-only glioblastoma tumor” is a tumor that cannot be resected. In some aspects, a biopsy-only glioblastoma tumor is a tumor that cannot be fully resected. For in example, in some aspects, a biopsy-only glioblastoma tumor is a tumor that can only be 1, 2, 3, 4, 5, 6, 7, 8, 9, 10, 12, 14, 16, 18, 20, 25, 30, 35, 40, 45, or 50% resected. In some aspects, a biopsy-only glioblastoma tumor is a tumor that cannot be resected at all. In some aspects, an inability to resect a biopsy-only glioblastoma tumor can be due to comorbid conditions or tumor locations involving eloquent regions of the brain. Thus, in some aspects, a biopsy-only glioblastoma tumor is a glioblastoma tumor that has not been and/or cannot be resected.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the lungs. In some aspects, a cell or population of cells that can be a target site or a target cell include, but are not limited to, a cancer cell (e.g., a lung cancer cell). In some aspects, a “target site” can be a tumor target site.

A “tumor target site” is a site or location within or present on a subject or patient that comprises or is adjacent to one or more non-small cell lung cancer cells, previously comprised one or more tumor cells, or is suspected of comprising one or more tumor cells. For example, a tumor target site can refer to a site or location within or present on a subject or patient that is prone to metastases (e.g. thorax). Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electric fields delivered to a subject, a sample obtained from a subject or to a specific location within a subject or patient (e.g. a target site). In some aspects, the alternating electrical field can be in a single direction or multiple directions. In some aspects, alternating electric fields can be delivered through two pairs of transducer arrays that generate perpendicular fields within the treated heart. For example, for the Optune™ system (an alternating electric fields delivery system) one pair of electrodes is located to the left and right (LR) of the heart, and the other pair of electrodes is located anterior and posterior (AP) to the heart. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of alternating electric fields therapy increases as the intensity of the electric field increases. Therefore, optimizing array placement on the area of a patient's tumor to increase the intensity in the desired region of the tumor can be performed with the Optune system. Array placement optimization may be performed by “rule of thumb” (e.g., placing the arrays on the tumor as close to the desired region of the target site (e.g. cancer cells) as possible), measurements describing the geometry of the patient's tumor, tumor dimensions. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electric field distributes within the head as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field and therapeutically effective amount of a checkpoint inhibitor.

By “treat” is meant to administer or apply a therapeutic, such as alternating electric fields, a checkpoint inhibitor, and/or temozolomide, to a subject, such as a human or other mammal (for example, an animal model), that has cancer or has an increased susceptibility for developing cancer, in order to prevent or delay a worsening of the effects of the cancer, or to partially or fully reverse the effects of the cancer (glioblastoma).

The term “prevent” can mean to minimize the chance a biopsy-only glioblastoma tumor will spread.

As used herein, the terms “administering” and “administration” refer to any method of providing a therapeutic, such as a checkpoint inhibitor to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a cancer cell to alternating electrical fields means administering alternating electrical fields to the cancer cell.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. As used herein, the term “therapeutically effective amount of a checkpoint inhibitor” means an amount of a therapeutic, prophylactic, and/or diagnostic checkpoint inhibitor that is sufficient, when administered in combination with an alternating electric field to a subject suffering from or susceptible to a disease (e.g. glioblastoma), disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying an alternating electric fields. In some aspects, the alternating electric field used in the methods disclosed herein is a tumor-treating field (TTFields). In some aspects, the alternating electric field can vary dependent on the type of cell or condition to which the alternating electric field is applied. In some aspects, the alternating electric field can be applied through one or more electrodes placed on or in the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be about 50 kHz, 100 kHz, 200 kHz, 300 kHz, 400 kHz, 500 kHz, or any frequency between. In some aspects, the frequency of the alternating electric field is from about 200 kHz to about 400 kHz, from about 250 kHz to about 350 kHz, and may be about 150 kHz, about 200 kHz, or about 300 kHz.

In some aspects, the field strength of the alternating electric fields can be between 1 and 4 V/cm RMS. In some aspects, different field strengths can be used (e.g., between 0.1 and 10 V/cm). In some aspects, the field strength can be about 1.75 V/cm RMS. In some embodiments the field strength is at least 1 V/cm. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two-hour duration.

In some aspects, the exposure may last for at least 6 hours, at least 12 hours, at least 24 hours, at least 36 hours, at least 48 hours, or at least 72 hours or more. In some aspects, the exposure can be consecutive or cumulative. In some aspects, the consecutive exposure may last for at least 6 hours, at least 12 hours, at least 24 hours, at least 36 hours, at least 48 hours, or at least 72 hours or more. In some aspects, the cumulative exposure may last for at least 42 hours, at least 84 hours, at least 168 hours, at least 250 hours, at least 400 hours, at least 500 hours, at least 750 hours, or more. In some aspects, there can be a break in treatment and the alternating electric fields are applied at least 50%, 60%, 70%, or 80% of treatment time. For example, in some aspects, cumulative exposure can be for at least 12 hours in a period of 24 hours.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

In some aspects, the exposure may last for at least 6 hours, at least 12 hours, at least 24 hours, at least 36 hours, at least 48 hours, or at least 72 hours or more.

In addition, when the alternating electric field is applied to a subject, the period of time that the alternating electric field is applied may be a continuous period of time or a cumulative period of time. That is, the period of time that the alternating electric field is applied may include a single session (i.e., continuous application) as well as multiple sessions with minor breaks in between sessions (i.e., consecutive applications for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.). For example, the alternating electric field can be applied for at least 12 hours, 16 hours, or 18 hours cumulative each day for a week, a month, two months, three months, etc.

### C. Methods of Treating

Disclosed are methods of treating a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject.

The methods disclosed herein comprise administering one or more checkpoint inhibitors to a subject. In some aspects, the checkpoint inhibitor can block CTLA-4 (cytotoxic T lymphocyte associated protein 4) PD-1 (programmed cell death protein 1) or PD-L1 (programmed cell death ligand 1).

In some aspects, the checkpoint inhibitor can be, but is not limited to, pembrolizumab (KEYTRUDA®), ipilimumab (YERVOY®), nivolumab (OPDIVO®), cemiplimab (LIBTAYO®), and dostarlimab (JEMPERLI), atezolizumab (TECENTRIQ®), durvalumab (IMFINZI®i), or avelumab (BAVENCIO®), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, the checkpoint inhibitor is pembrolizumab (KEYTRUDA®). In some aspects, Pembrolizumab can be administered at a dose of 200 mg. In some aspects, Pembrolizumab can be administered at a dose of 100 mg to 500 mg. For example, in some aspects, Pembrolizumab can be administered at a dose of 200 mg every three weeks starting at the second round, or cycle, of alternating electric fields and TMZ.

In some aspects, the methods pertain to a subject having a biopsy-only glioblastoma tumor who was previously treated with a checkpoint inhibitor before the combination treatment of alternating electric field, TMZ and checkpoint inhibitor. In such embodiments, the checkpoint inhibitor can be an inhibitor that blocks CTLA-4 (cytotoxic T lymphocyte associated protein 4) PD-1 (programmed cell death protein 1) or PD-L1 (programmed cell death ligand 1). In some aspects, the checkpoint inhibitor previously administered to the subject can be, but is not limited to, ipilimumab (YERVOY®), pembrolizumab (KEYTRUDA®), nivolumab (OPDIVO®), cemiplimab (LIBTAYO®), and dostarlimab (JEMPERLI), atezolizumab (TECENTRIQ®), durvalumab (IMFINZI®i), or avelumab (BAVENCIO®), or a combination thereof. In some aspects, the checkpoint inhibitor can be, but is not limited to, tremelimumab, sintilimab (formerly IBI308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof. Thus, in some aspects, the subject can have failed an initial treatment with checkpoint inhibitor.

In some aspects of the disclosed methods, applying an alternating electric field occurs 1, 2, 3, 4, 5, 6, or 7 days prior to administering the TMZ and/or checkpoint inhibitor. In some aspects, applying an alternating electric field occurs 1, 2, 3, 4, 5, 6, or 7 days after administering the TMZ and/or checkpoint inhibitor. In some aspects, applying alternating electric fields occurs 1, 2, 3, or 4 weeks prior to administering the TMZ and/or checkpoint inhibitor. In some aspects, applying alternating electric fields occurs 1, 2, 3, or 4 weeks after administering the TMZ and/or checkpoint inhibitor. In some aspects, the alternating electric fields and one or both of the TMZ and the checkpoint inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the TMZ and/or checkpoint inhibitor are present in the bloodstream prior to applying the alternating electric field.

In some aspects, the disclosed methods further comprise discontinuing the alternating electric field during the method. In some aspects, the alternating electric field can be applied discontinuously over the course of treatment. For example, the alternating electric field can be applied less than 24 hours a day and 7 days a week. In some aspects, the alternating electric field can be applied at least 6, 7, 8, 9, 10, 11, 12, 13, 14, 15, 16, 17, 18 or 19 hours a day or more.

In some aspects, the alternating electric field is administered prior to the TMZ and checkpoint inhibitor. In some aspects, the TMZ is administered prior to the alternating electric field and checkpoint inhibitor. In some aspects, the checkpoint inhibitor is administered prior to the alternating electric field and TMZ. In some aspects, the checkpoint inhibitor is administered after the alternating electric field and TMZ. In some aspects, the alternating electric field, TMZ and checkpoint inhibitor are administered simultaneously.

In some aspects, the TMZ is administered for a period of time prior to the alternating electric field and checkpoint inhibitor. In some aspects, after an initial dosing with TMZ, a combination of the alternating electric field and TMZ (i.e., adjuvant TMZ) can be administered for a period of time. In some aspects, the period of time of administering the alternating electric field and TMZ can be at least for one cycle all the way up to 12 cycles, wherein a single cycle can be a month.

In some aspects, after treatment with the combination of the alternating electric field and TMZ, the checkpoint inhibitor can be administered for a period of time, wherein all three of the alternating electric field, TMZ, and the checkpoint inhibitor are administered simultaneously for a period of time. In some aspects, the checkpoint inhibitor is administered after one cycle of alternating electric field and TMZ. In some aspects, the period of time of administering the checkpoint inhibitor is every three weeks beginning on day 1 of cycle 2 of the alternating electric field and TMZ treatment. In some aspects, after administering all three of the alternating electric field, TMZ, and the checkpoint inhibitor, the TMZ can be stopped and only the alternating electric field and checkpoint inhibitor are administered for a period of time. For example, in some aspects, the combination treatment with all three of the alternating electric field, TMZ, and the checkpoint inhibitor can be stopped after 6, 7, 8, 9, 10 or 12 months and only the alternating electric field and checkpoint inhibitor are administered for the remaining months out to a total of 24 months of total treatment time with the alternating electric field.

In some aspects, the initial dosing with TMZ prior to treatment with the alternating electric field can be administered concomitantly with radiation therapy. In some aspects, four to six weeks after the chemoradiation, subjects can start monthly cycles of adjuvant TMZ. Treatment with alternating electric fields can start at approximately the same time as the first cycle of adjuvant TMZ. In some aspects, the alternating electric field and TMZ treatment can continue until second disease progression or a maximum of 2 years. In some aspects, a minimum of 6 and maximum of 12 cycles of adjuvant TMZ can be administered. In some aspects, within one week after starting cycle 2 of adjuvant TMZ and the alternating electric field therapy, subjects can begin treatment with a checkpoint inhibitor, such as Pembrolizumab, every 3 weeks until first disease progression or unacceptable toxicities or 2 years, whichever comes first. In some aspects, the checkpoint inhibitor, such as Pembrolizumab, can be given intravenously every 3 weeks beginning on day 1 of cycle 2 of adjuvant TMZ. Treatment with the checkpoint inhibitor (e.g., Pembrolizumab) every 3 weeks until first disease progression or unacceptable toxicities or 2 years, whichever comes first.

In some aspects, the methods can follow the known 2-THE-TOP clinical trial regimen wherein the subject is one having a biopsy-only glioblastoma tumor.

In some aspects, the subject has previously undergone standard of care TMZ treatment and/or radiation therapy prior to treatment with the combination of alternating electric field, TMZ and checkpoint inhibitor. Thus, the TMZ in the combination of alternating electric field, TMZ and checkpoint inhibitor can be referred to an adjuvant TMZ.

In some aspects, the alternating electric field can have a frequency and field strength. In some aspects, the frequency of the alternating electric field is between 50 kHz and 1 MHz. In some aspects, the frequency of the alternating electric field is 100 kHz-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some aspects of the disclosed methods of treating, antigen-specific T cell stimulation is increased in the subject. In some aspects, antigen-specific T cell stimulation is increased in the subject after at least cycle 2 of the alternating electric field and TMZ, which is equivalent to cycle 1 of the combination of the alternating electric field, TMZ, and a checkpoint inhibitor.

In some aspects of the disclosed methods of treating, T cell receptor (TCR) clonal turnover is increased in the subject. In some aspects, TCR clonal turnover is increased in the subject after at least cycle 2 of the alternating electric field and TMZ, which is equivalent to cycle 1 of the combination of the alternating electric field, TMZ, and a checkpoint inhibitor.

In some aspects of the disclosed methods of treating, central memory T cell development is increased in the subject. In some aspects, central memory T cell development is increased in the subject after at least cycle 2 of the alternating electric field and TMZ, which is equivalent to cycle 1 of the combination of the alternating electric field, TMZ, and a checkpoint inhibitor.

In some aspects of the disclosed methods of treating, the increase of antigen-specific T cell stimulation and/or T cell receptor (TCR) clonal turnover and/or central memory T cell development is higher in a biopsy-only subject compared to a subject having maximal tumor resection.

In some aspects, a subject with biopsy-only glioblastoma tumors has improved progression-free survival, overall survival, and response rates compared to a subject who underwent maximal tumor resection. In some aspects, the improvement in progression-free survival, overall survival, and response rates is after at least cycle 2 of the alternating electric field and TMZ, which is equivalent to cycle 1 of the combination of the alternating electric field, TMZ, and a checkpoint inhibitor.

In some aspects, CD4+ T cells are the predominant T cell subtype undergoing robust clonal replacement. In some aspects, there is a combination of CD8+ and CD4+ T cells undergoing robust clonal replacement. In some aspects, the clonal replacement is after at least cycle 2 of the alternating electric field and TMZ, which is equivalent to cycle 1 of the combination of the alternating electric field, TMZ, and a checkpoint inhibitor.

In some aspects, the disclosed methods of treating further comprise determining the presence of CD4+ or CD8+ clonal replacement after treatment with the alternating electric field, TMZ and checkpoint inhibitor. In some aspects, the clonal replacement can be compared to a standard or known amount that naturally occurs without treating or with treatment of just one of the alternating electric field, TMZ and checkpoint inhibitor. In some aspects, the clonal replacement can be compared to an amount determined prior to treatment with the alternating electric field, TMZ and checkpoint inhibitor. In some aspects, an increase in CD4+ or CD8+ clonal replacement indicates the treatment is effective. In some aspects, a decrease in CD4+ or CD8+ clonal replacement indicates treatment with the alternating electric field, TMZ and checkpoint inhibitor should be stopped.

### D. Methods of Increasing Survival

Disclosed are methods of increasing survival of a subject having a biopsy-only glioblastoma tumor comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject.

The methods disclosed herein comprise administering one or more checkpoint inhibitors to a subject. In some aspects, the checkpoint inhibitor can block CTLA-4 (cytotoxic T lymphocyte associated protein 4) PD-1 (programmed cell death protein 1) or PD-L1 (programmed cell death ligand 1).

In some aspects, the checkpoint inhibitor can be, but is not limited to, pembrolizumab (Keytruda), ipilimumab (Yervoy), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, the checkpoint inhibitor is pembrolizumab (Keytruda). In some aspects, Pembrolizumab can be administered at a dose of 200 mg. In some aspects, Pembrolizumab can be administered at a dose of 100 mg to 500 mg. For example, in some aspects, Pembrolizumab can be administered at a dose of 200 mg every three weeks starting at the second round, or cycle, of alternating electric fields and TMZ.

In some aspects, the methods pertain to a subject having a biopsy-only glioblastoma tumor who was previously treated with a checkpoint inhibitor before the combination treatment of alternating electric field, TMZ and checkpoint inhibitor. In such embodiments, the checkpoint inhibitor can be an inhibitor that blocks CTLA-4 (cytotoxic T lymphocyte associated protein 4) PD-1 (programmed cell death protein 1) or PD-L1 (programmed cell death ligand 1). In some aspects, the checkpoint inhibitor previously administered to the subject can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, tremelimumab, sintilimab (formerly IBI308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof. Thus, in some aspects, the subject can have failed an initial treatment with checkpoint inhibitor.

In some aspects of the disclosed methods, applying an alternating electric field occurs 1, 2, 3, 4, 5, 6, or 7 days prior to administering the TMZ and/or checkpoint inhibitor. In some aspects, applying an alternating electric field occurs 1, 2, 3, 4, 5, 6, or 7 days after administering the TMZ and/or checkpoint inhibitor. In some aspects, applying alternating electric fields occurs 1, 2, 3, or 4 weeks prior to administering the TMZ and/or checkpoint inhibitor. In some aspects, applying alternating electric fields occurs 1, 2, 3, or 4 weeks after administering the TMZ and/or checkpoint inhibitor. In some aspects, the alternating electric fields and one or both of the TMZ and the checkpoint inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the TMZ and/or checkpoint inhibitor are present in the bloodstream prior to applying the alternating electric field.

In some aspects, the disclosed methods further comprise discontinuing the alternating electric field during the method. In some aspects, the alternating electric field can be applied discontinuously over the course of treatment. For example, the alternating electric field can be applied less than 24 hours a day and 7 days a week. In some aspects, the alternating electric field can be applied at least 6, 7, 8, 9, 10, 11, 12, 13, 14, 15, 16, 17, 18 or 19 hours a day or more.

In some aspects, the alternating electric field is administered prior to the TMZ and checkpoint inhibitor. In some aspects, the TMZ is administered prior to the alternating electric field and checkpoint inhibitor. In some aspects, the checkpoint inhibitor is administered prior to the alternating electric field and TMZ. In some aspects, the checkpoint inhibitor is administered after the alternating electric field and TMZ. In some aspects, the alternating electric field, TMZ and checkpoint inhibitor are administered simultaneously.

In some aspects, the TMZ is administered for a period of time prior to the alternating electric field and checkpoint inhibitor. In some aspects, after an initial dosing with TMZ, a combination of the alternating electric field and TMZ (i.e., adjuvant TMZ) can be administered for a period of time. In some aspects, the period of time of administering the alternating electric field and TMZ can be at least for one cycle all the way up to 12 cycles, wherein a single cycle can be a month.

In some aspects, after treatment with the combination of the alternating electric field and TMZ, the checkpoint inhibitor can be administered for a period of time, wherein all three of the alternating electric field, TMZ, and the checkpoint inhibitor are administered simultaneously for a period of time. In some aspects, the checkpoint inhibitor is administered after one cycle of alternating electric field and TMZ. In some aspects, the period of time of administering the checkpoint inhibitor is every three weeks beginning on day 1 of cycle 2 of the alternating electric field and TMZ treatment. In some aspects, after administering all three of the alternating electric field, TMZ, and the checkpoint inhibitor, the TMZ can be stopped and only the alternating electric field and checkpoint inhibitor are administered for a period of time. For example, in some aspects, the combination treatment with all three of the alternating electric field, TMZ, and the checkpoint inhibitor can be stopped after 6, 7, 8, 9, 10 or 12 months and only the alternating electric field and checkpoint inhibitor are administered for the remaining months out to a total of 24 months of total treatment time with the alternating electric field.

In some aspects, the initial dosing with TMZ prior to treatment with the alternating electric field can be administered concomitantly with radiation therapy. In some aspects, four to six weeks after the chemoradiation, subjects can start monthly cycles of adjuvant TMZ. Treatment with alternating electric fields can start at approximately the same time as the first cycle of adjuvant TMZ. In some aspects, the alternating electric field and TMZ treatment can continue until second disease progression or a maximum of 2 years. In some aspects, a minimum of 6 and maximum of 12 cycles of adjuvant TMZ can be administered. In some aspects, within one week after starting cycle 2 of adjuvant TMZ and the alternating electric field therapy, subjects can begin treatment with a checkpoint inhibitor, such as Pembrolizumab, every 3 weeks until first disease progression or unacceptable toxicities or 2 years, whichever comes first. In some aspects, the checkpoint inhibitor, such as Pembrolizumab, can be given intravenously every 3 weeks beginning on day 1 of cycle 2 of adjuvant TMZ. Treatment with the checkpoint inhibitor (e.g., Pembrolizumab) every 3 weeks until first disease progression or unacceptable toxicities or 2 years, whichever comes first.

In some aspects, the methods can follow the known 2-THE-TOP clinical trial regimen wherein the subject is one having a biopsy-only glioblastoma tumor.

In some aspects, the subject has previously undergone standard of care TMZ treatment and/or radiation therapy prior to treatment with the combination of alternating electric field, TMZ and checkpoint inhibitor. Thus, the TMZ in the combination of alternating electric field, TMZ and checkpoint inhibitor can be referred to an adjuvant TMZ.

In some aspects, the alternating electric field can have a frequency and field strength. In some aspects, the frequency of the alternating electric field is between 50 kHz and 1 MHz. In some aspects, the frequency of the alternating electric field is 100 kHz-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some aspects of the disclosed methods, antigen-specific T cell stimulation is increased in the subject. In some aspects, antigen-specific T cell stimulation is increased in the subject after at least cycle 2 of the alternating electric field and TMZ, which is equivalent to cycle 1 of the combination of the alternating electric field, TMZ, and a checkpoint inhibitor.

In some aspects of the disclosed methods, T cell receptor (TCR) clonal turnover is increased in the subject. In some aspects, TCR clonal turnover is increased in the subject after at least cycle 2 of the alternating electric field and TMZ, which is equivalent to cycle 1 of the combination of the alternating electric field, TMZ, and a checkpoint inhibitor.

In some aspects of the disclosed methods of treating, central memory T cell development is increased in the subject. In some aspects, central memory T cell development is increased in the subject after at least cycle 2 of the alternating electric field and TMZ, which is equivalent to cycle 1 of the combination of the alternating electric field, TMZ, and a checkpoint inhibitor.

In some aspects of the disclosed methods, the increase of antigen-specific T cell stimulation and/or T cell receptor (TCR) clonal turnover and/or central memory T cell development is higher in a biopsy-only subject compared to a subject having maximal tumor resection.

In some aspects, a subject with biopsy-only glioblastoma tumors has improved progression-free survival, overall survival, and response rates compared to a subject who underwent maximal tumor resection. In some aspects, the improvement in progression-free survival, overall survival, and response rates is after at least cycle 2 of the alternating electric field and TMZ, which is equivalent to cycle 1 of the combination of the alternating electric field, TMZ, and a checkpoint inhibitor.

In some aspects, CD4+ T cells are the predominant T cell subtype undergoing robust clonal replacement. In some aspects, there is a combination of CD8+ and CD4+ T cells undergoing robust clonal replacement. In some aspects, the clonal replacement is after at least cycle 2 of the alternating electric field and TMZ, which is equivalent to cycle 1 of the combination of the alternating electric field, TMZ, and a checkpoint inhibitor.

In some aspects, the disclosed methods further comprise determining the presence of CD4+ or CD8+ clonal replacement after treatment with the alternating electric field, TMZ and checkpoint inhibitor. In some aspects, the clonal replacement can be compared to a standard or known amount that naturally occurs without treating or with treatment of just one of the alternating electric field, TMZ and checkpoint inhibitor. In some aspects, the clonal replacement can be compared to an amount determined prior to treatment with the alternating electric field, TMZ and checkpoint inhibitor. In some aspects, an increase in CD4+ or CD8+ clonal replacement indicates the treatment is effective. In some aspects, a decrease in CD4+ or CD8+ clonal replacement indicates treatment with the alternating electric field, TMZ and checkpoint inhibitor should be stopped.

### Examples

### 1. Introduction

Immunotherapies, including immune checkpoint inhibitors (ICIs) like anti-PD-1/PD-L1 monoclonal antibodies, have shown high benefit for many solid tumors. However, their effectiveness in GBM remains limited, despite the significant expression of the PD-1/PD-L1 axis in these tumors. The challenges in developing new immunotherapeutic approaches for GBM are multifaceted, involving the tumor's low mutation burden, extensive molecular heterogeneity, and an immunosuppressive or “cold” tumor microenvironment (TME). This TME is deficient in T cells and dendritic cells but replete with immunosuppressive cell populations, such as regulatory T cells (Tregs) and myeloid-derived suppressor cells (MDSCs), along with signals that facilitate immune escape. Current strategies focused on mobilizing systemic cytotoxic T cell responses have been met with variable success, indicating that potent peripheral immune activation may not suffice to modulate the cold TME to synergistically enhance the efficacy of ICIs. Consequently, recent seminal research has pivoted towards directly targeting the TME. This includes the use of intraoperative, intracavitary, or implantable reservoirs for the local delivery of therapies such as hyperthermic treatments, oncolytic viruses, or gene therapy to elicit in situ vaccination effects, with some approaches showing encouraging results when used in combination with ICIs. Nonetheless, there is a critical need for the development of non-invasive strategies capable of directly modulating the TME of GBM. Such strategies should allow for safe, repeated administration to achieve consistent and sustained TME stimulation.

TTFields, a non-invasive modality utilizing low-intensity, intermediate-frequency alternating electric fields, have elicited notable anti-neoplastic effects via a plethora of cellular and molecular mechanisms. The therapeutic application of TTFields has demonstrated excellent tolerability and survival extension, culminating in its approval for the treatment of GBM and malignant pleural mesothelioma. Recent investigative efforts have concentrated on TTFields' capacity to initiate immunogenic cell death (ICD), augment the TME's permeability to immune effector cells, and preserve T lymphocyte functionality, thereby implicating a significant impact on modulating the immune TME of GBM. From a mechanistic standpoint, recent studies have revealed that TTFields application induces discrete disruptions within the nuclear envelope of GBM and other solid tumor cells, precipitating the cytosolic dissemination of large clusters of naked DNA. This phenomenon actuates key DNA sensing pathways and their associated inflammasomes, specifically cGAS/STING and AIM2/Caspase-1, leading to the production of copious type I interferons (T1IFN) and pro-inflammatory cytokines. Concurrently, TTFields engender programmed necrotic ICD, releasing tumor immunogens and thereby creating a non-invasive, on-demand, in situ immunization construct against GBM and, potentially, other solid tumors. In patients with newly diagnosed GBM, TTFields therapy has been correlated with robust adaptive immune system engagement, as evidenced by marked T cell receptor (TCR) clonal expansion and T cell activation, predominantly via a T1IFN trajectory.

To investigate the potential synergistic effects of TTFields and the anti-PD-1 immunotherapy pembrolizumab, along with adjuvant temozolomide (TMZ), a pilot study was conducted involving patients with newly diagnosed GBM following either maximal tumor resection or biopsy only and completion of standard concomitant TMZ and radiotherapy. The objective was to corroborate TTFields' capacity for in situ vaccination and reheating the TME by assessing clinical outcomes and immune dynamics, particularly in patients with bulky, biopsy-only tumors. While these biopsy-only patients typically carry the most dismal prognosis, they conceivably possess an increased neoplastic burden amenable to the immunizing effects of TTFields, relative to those who have undergone maximal tumor excision. A multi-faceted analytical approach was used, employing T cell receptor (TCR) sequencing, bulk RNA sequencing (RNA-seq) of enriched T cell populations, targeted single cell RNA-seq (scRNA-seq) alongside multiplex immunohistochemistry (IHC) on primary and recurrent tumor specimens to delineate the molecular determinants and mechanism of response.

### 2. Results

i. Study Design, Patient Demographics and Baseline Characteristics.

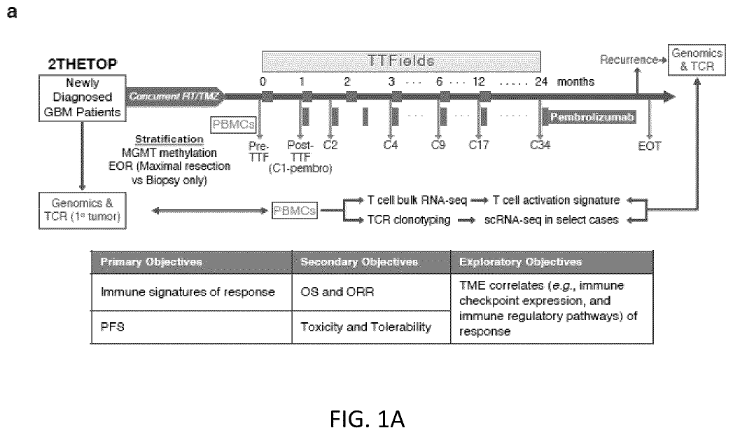

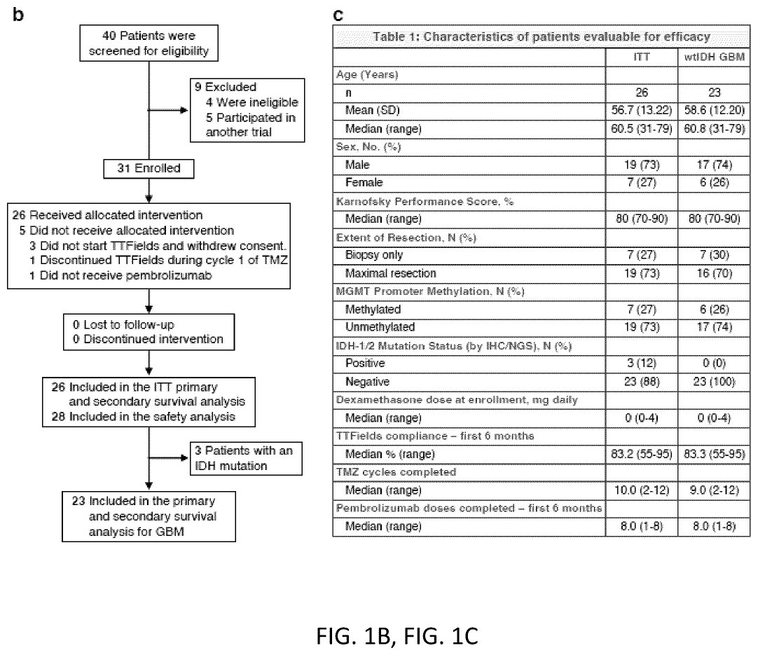

To investigate the putative synergistic effects of TTFields' inherent in situ vaccination properties with ICIs, a Phase 2 pilot trial was initiated combining TTFields with pembrolizumab, a PD-1 blocking antibody, and adjuvant TMZ in patients with newly diagnosed GBM (study's acronym: 2THETOP), who had undergone either maximal tumor resection or biopsy only due to comorbid conditions or tumor locations involving eloquent regions of the brain. All eligible patients must have completed standard radiation and concurrent TMZ, had good performance status (i.e., KPS of 70%) with adequate hematologic and metabolic reserves, and required no more than 4 mg daily of dexamethasone. Administration of TTFields commenced concurrently with the initiation of adjuvant TMZ therapy. Pembrolizumab, dosed at 200 mg intravenously every 3 weeks, was introduced starting with the second TMZ cycle. This staged approach was strategically chosen to facilitate the delineation of immunological effects attributable to TTFields from those synergistically induced by the combined regimen of TTFields and pembrolizumab. The elucidation of immune response signatures and their association with progression-free survival (PFS) were the primary study objectives (FIG. 1a). Disease assessment was performed using the Immunotherapy Response Assessment in Neuro-Oncology (iRANO) criteria. Secondary objectives included overall survival (OS), objective response rate, and safety. Additionally, an exploratory objective was to identify molecular TME markers correlating with therapeutic response.

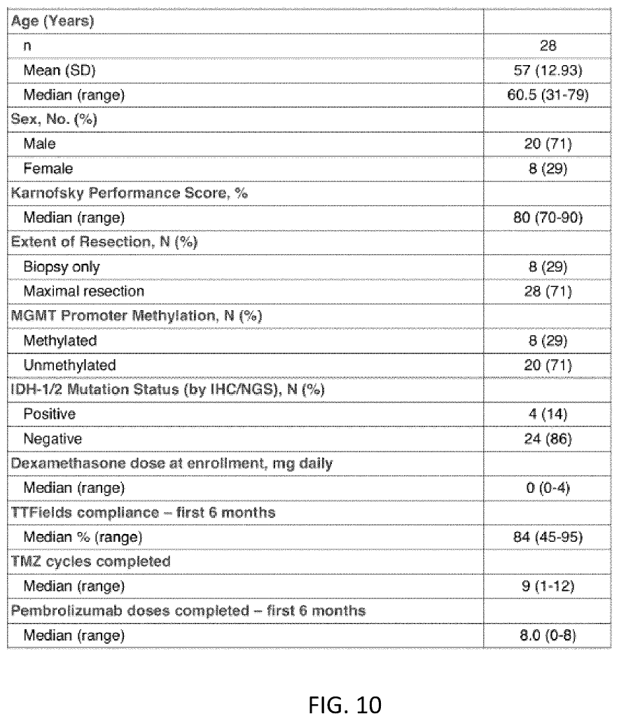

From 2018 to 2021, 31 eligible patients were enrolled (FIG. 1b). Five patients did not start assigned study treatments. Of these 5 patients, 3 did not start TTFields and withdrew consent to the study, and 2 started TTFields but discontinued during the first month of treatment and did not receive at least 1 cycle of pembrolizumab, which was required for evaluability for survival and immune signature analysis. However, these 2 patients were included in the safety analysis. Basic characteristics of patients evaluable for safety and efficacy are detailed in FIG. 10 and FIG. 1c, respectively. For the 26 patients eligible for efficacy analysis, the median age was 60.5 years and 73% were men. Seven patients (27%) had biopsy only, 19 (73%) with an unmethylated MGMT promoter, and 3 (12%) with an IDH1/2 mutation as determined by IHC and DNA sequencing. During the accrual period, important updates to the World Health Classification (WHO) of CNS tumors were instituted in the field that separated IDH1/2 mutant tumors from the GBM classification to establish a distinct subtype of WHO grade IV astrocytoma with significantly better prognosis compared to classical GBM. As a result, for the last 25% enrollments, only wild-type IDH1/2 (wtIDH) GBM were enrolled. To minimize this subtype selection bias, a wtIDH GBM only population (23 patients) was also created for analysis alongside the intent-to-treat (ITT) population of 26 patients (FIG. 1c).

ii. Safety

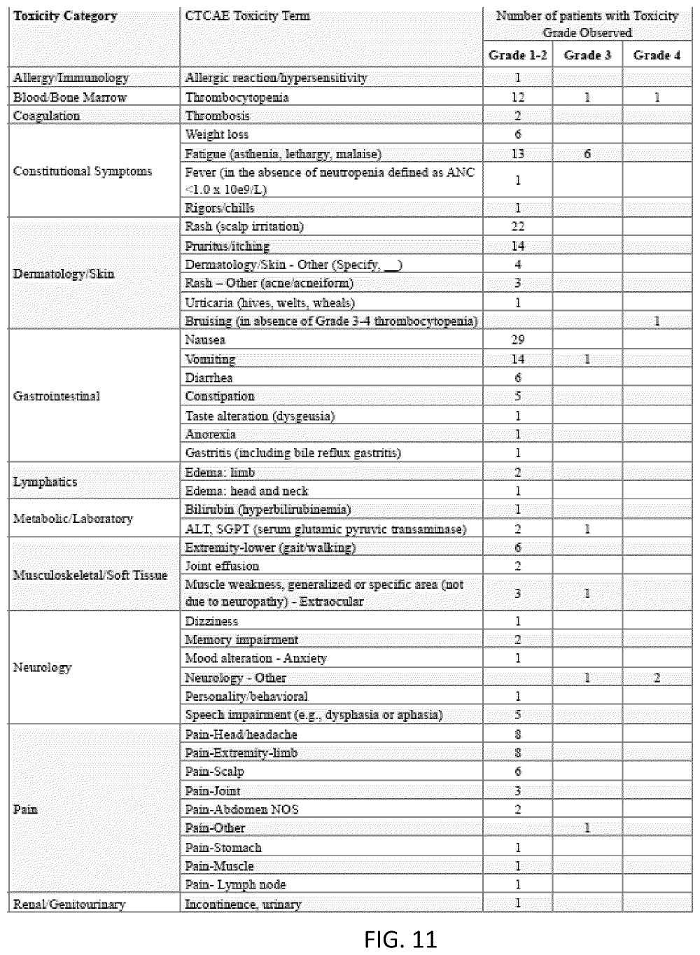

Throughout the pilot trial, all the adverse events were meticulously monitored and documented. Among the recorded events, treatment-related adverse events, specifically 213, constituted 31% of the total of 695. The toxicity table (FIG. 11) offers a comprehensive overview of these 213 potential toxicity events with the investigational treatment. The most frequently reported adverse events were dermatology-related—especially scalp irritation from TTFields array placement—and gastrointestinal—the most common being nausea—with the majority being mild to moderate in severity. Only 16 out of these 213 recorded events, approximately 7.5%, were classified as CTCAE grade above 2. The trial demonstrated an overall manageable safety profile, with very infrequent occurrences of severe adverse events that were promptly addressed. These findings instill confidence in the safety and tolerability of the triple combination (TTFields, pembrolizumab, and TMZ), establishing a foundation for progression to further clinical development.

iii. Patients with Biopsy-Only Tumors have Higher Objective Response and Survival Rates.

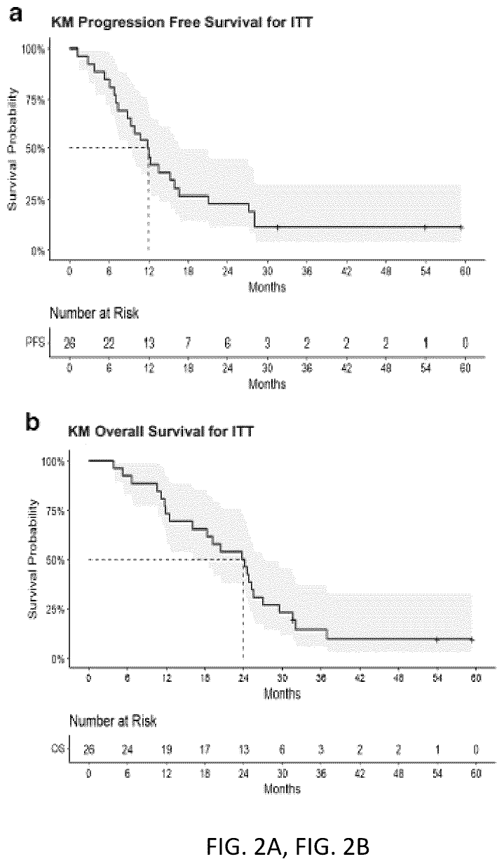

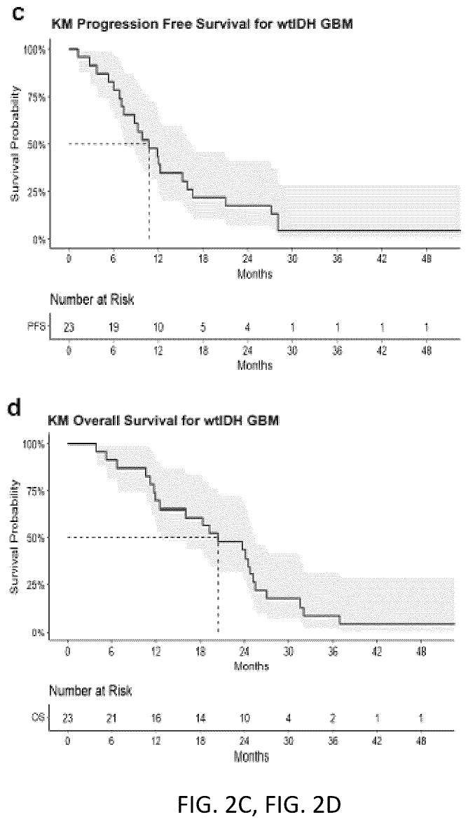

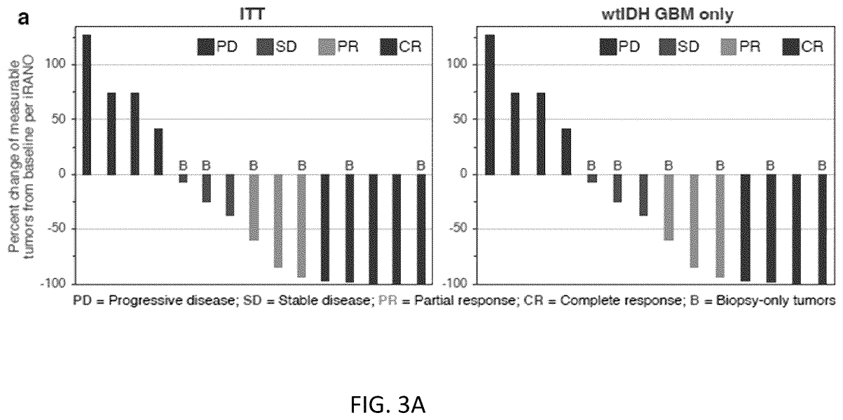

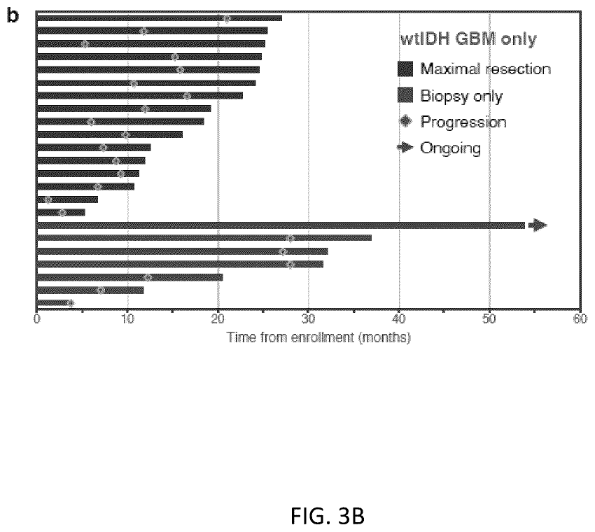

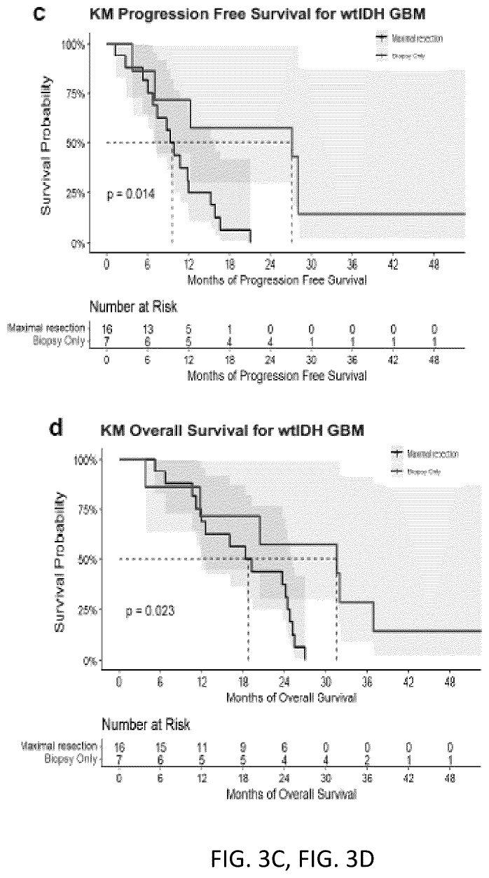

Twenty-six patients completed at least one dose of pembrolizumab and were included in efficacy analysis as stipulated by the protocol. A summary of the efficacy analysis is presented in Table 1. As of data analytical cut-off date, 3 patients had not progressed and were live. The 26-patient ITT GBM population with 3 IDH mutant tumors reached median PFS of 11.9 months (95% CI, 8.83-21.1 months) and median OS of 24.0 months (95% CI, 16.1-29.5 months), despite being enriched in several poor prognostic features (i.e., 73% males, 27% biopsy only, and 73% unmethylated MGMT promoter) (FIGS. 1c and 2a-b). The median PFS and median OS of the 23-patient wtIDH GBM only population were 10.8 months (95% CI, 7.4-16.6 months) and 20.5 months (95% CI, 12.5-25.5 months), respectively (FIG. 2c-d). In 15 patients with a measurable target tumor in the ITT population, per iRANO) criteria, 4 patients had a complete response (CR) and 3 with a partial response (PR) for an overall objective response rate of 46.7% (95% CI, 24.8-69.9%). The progressive disease (PD) and stable disease (SD) rates of the ITT population were 53.8% (95% CI, 35.5-71.2%) and 19.2% (95% CI, 8.5-37.9%), respectively. In the wtIDH GBM only population, the response rate, PD, and SD rates were 42.8% (95% CI, 21.4-67.4%), 60.9% (95% CI, 36.8-74.4%), and 13.0% (95% CI, 4.5-32.1%), respectively (Table 1 and FIG. 3a).

TABLE 1
Characteristics of Maximal-resection and Biopsy-only Groups and Summary of Efficacy

| | | | | wild-type IDH1/2 GBM only | | |
|---|---|---|---|---|---|---|
| | Intent-to-treat population, N = 26 | | | population, N = 23 | | |
| | | Maximal | | | Maximal | |
| Evaluable | | resection, | Biopsy only, | | resection, | Biopsy only, |
| subjects, N (%) | 26 | 19 (73%) | 7 (27%) | 23 | 16 (70%) | 7 (30%) |
| IDH1/2 mutation, | 3 | 3 | 0 | 0 | 0 | 0 |
| N (%) | (11.5%) | (15.8%) | | | | |
| Unmethylated | 19 | 14 | 5 | 17 | 12 | 5 |
| MGMT, N (%) | (73%) | (74%) | (71%) | (74%) | (75%) | (71%) |
| ORR, % (95% CI) | 46.7% | 33.3% | 66.6% | 42.8% | 25% | 66.6% |
| | (24.8-69.9%) | (12.1-64.6%) | (30.0-90.3%) | (21.4-67.4%) | (7.1-59.1%) | (30.0-90.3%) |
| CR, % (95% CI) | 26.7% | 22.2%. | 33.3% | 21.4% | 12.5% | 33.3% |
| | (10.9-51.9%) | (6.3-54.7%) | (9.7-70%) | (7.6-47.6%) | (2.2-47.1%) | (9.7-70%) |
| PR, % (95% CI) | 20% | 11.1% | 33.3% | 21.4% | 12.5% | 33.3% |
| | (7.0-45.2%) | (2.0-43.5%) | (9.7-70%) | (7.6-47.6%) | (2.2-47.1%) | (9.7-70%) |
| PD, % (95% CI) | 53.8% | 68.4% | 14.3% | 60.9% | 81.2% | 14.3% |
| | (35.5-71.2%) | (46.0-84.6%) | (2.6-51.3%) | (36.8-74.4%) | (57.0-93.4%) | (2.6-51.3%) |
| SD, % (95% CI) | 19.2% | 15.8% | 28.6% | 13.0% | 6.3% | 28.6% |
| | (8.5-37.9%) | (5.5-37.6%) | (8.2-64.1%) | (4.5-32.1%) | (1.1-28.3%) | (8.2-64.1%) |
| Survival | | | | | | |
| PFS (months) | 12.0 | 10.8 | 27.2 | 10.8 | 9.6 | 27.2 |
| HR (95% CI); P | | 0.58 (0.25-1.34); P = 0.231 | | | 0.37 (0.16-0.85); P = 0.0137 | |
| OS (months) | 24.0 | 23.7 | 31.6 | 20.5 | 18.8 | 31.6 |

| | | |
|---|---|---|
| HR (95% CI); P | 0.59 (0.25-1.38); P = 0.349 | 0.4 (0.17-0.92); P = 0.0233 |

More importantly, patients with biopsy-only tumors in either the ITT or wtIDH GBM only population achieved a response rate of 66.6% (95% CI, 30.0-90.3%), while their PD rate was only 14.3% (95% CI, 2.6-51.3%), compared to 33.3% (95% CI, 12.1-64.6%) and 68.4% (95% CI, 46.0-84.6%), respectively, in the maximal resection group of the ITT population, and 25% (95% CI, 7.1-59.1%) and 81.2% (95 CI, 57.0-93.4%), respectively, in the maximal resection group of the wtIDH GBM only population (Table 1). Although the significantly higher response rate and lower PD rate in patients with biopsy-only tumors were not anticipated based on the extensive historical observations associating poorer prognosis and outcomes with biopsy-only tumors, they are in keeping with previous findings establishing TTFields as a complete in situ immunizing platform for GBM-presumably the presence of large tumors may provide the necessary tumor bulk for the in situ vaccination effects to materialize.

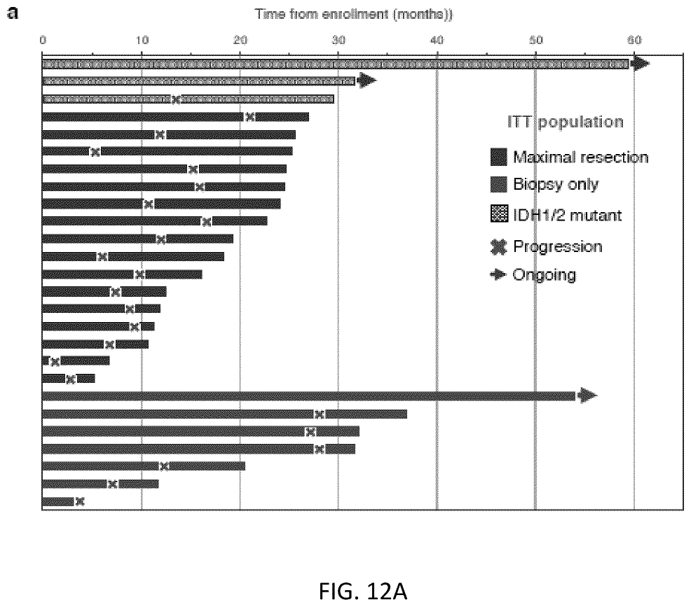

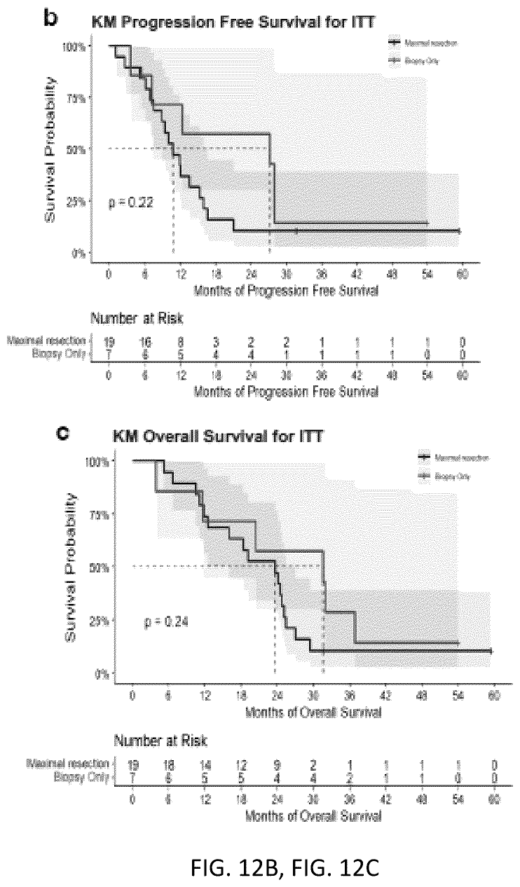

Consistent with this notion, patients with biopsy-only tumors in the wtIDH GBM only population exhibited greatly extended survival benefits compared to those with maximal resection in both median PFS (27.2 months vs. 9.6 months; HR 0.37; 95% CI, 0.16-0.85; log rank P=0.0137) and median OS (31.2 months vs. 18.8 months; HR 0.4; 95% CI, 0.17-0.92; log rank P=0.0233) (Table 1 and FIG. 3b-d). In the ITT population, despite with 3 IDH1/2 mutant patients exclusively in the maximal resection cohort, biopsy-only patients still trended toward surpassing maximal resection patients in both median PFS (27.2 months vs 10.8 months; HR 0.58; 95% CI, 0.25-1.34) and median OS (31.2 months vs 23.7 months; HR 0.59; 95% CI, 0.25-1.38) milestones (Table 1 and FIG. 12).

iv. The Immune TME in Primary Maximal Resection and Biopsy-Only Tumors Shared Similarities.

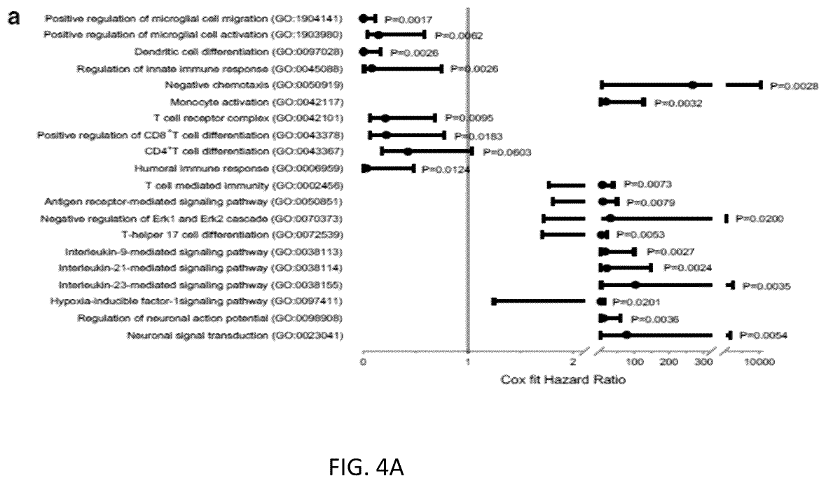

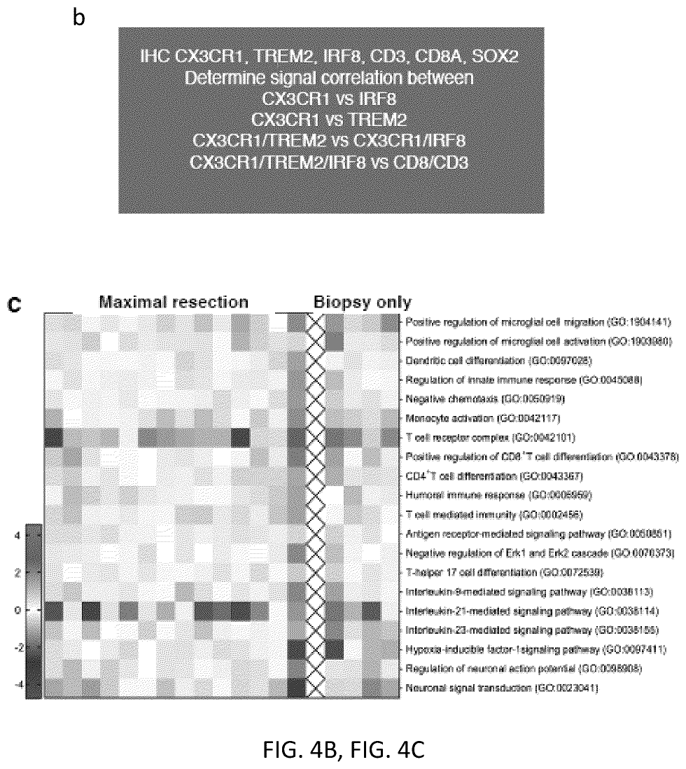

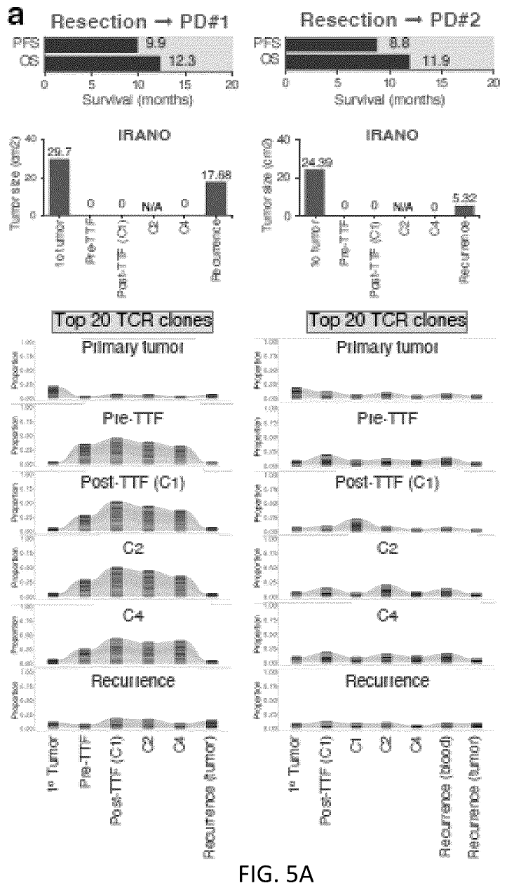

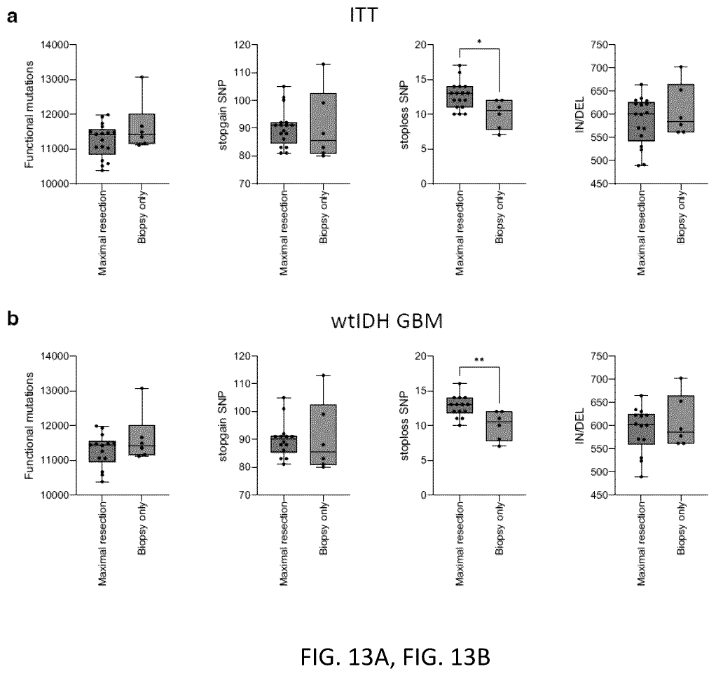

To determine if the observed survival benefit in patients with biopsy-only tumors was coincidental or influenced by intrinsic TME characteristics, available primary tumors were analyzed from 14 (of 19) maximal resection and 6 (of 7) biopsy-only cases. The evaluation focused on tumor mutational burden (TMB) via whole exome sequencing and immune TME profiles using bulk RNA-seq. TMB is known to correlate with responses to immune checkpoint inhibitors (ICIs) in solid tumors. Comparative analysis did not reveal significant disparities in functional TMB, stop-gain single-nucleotide polymorphisms (SNPs), or insertions/deletions between the 2 groups. A marginal increase in stop-loss SNPs was noted in the maximally resected tumors, however (FIG. 13). Consistent with GBM's immunosuppressive TME, upregulated pathways were observed from the Gene Ontology collection, implicated in MDSC recruitment, activation, and retention, as well as those engaged in hypoxic responses and neuronal activities, which potentially facilitate GBM progression and glioma stem-like cells (GSC). In a multivariate Cox Proportional HR analysis incorporating established prognostic factors and survival data, these pathways were associated with poorer clinical outcomes (FIG. 4a). Consistent with TILs being linked to enhanced responses to immunotherapies, including ICIs, the study also indicated that the presence of TILs, as demonstrated by TCR complex and CD4⁺ and CD8⁺ T cell differentiation markers, correlated with better patient outcomes. Nevertheless, the signaling pathways of these TILs did not mirror this positive association, which aligns with current understandings that TILs in GBM are predominantly in a dysfunctional state, contributing to tumor-promoting activities rather than antitumor responses.

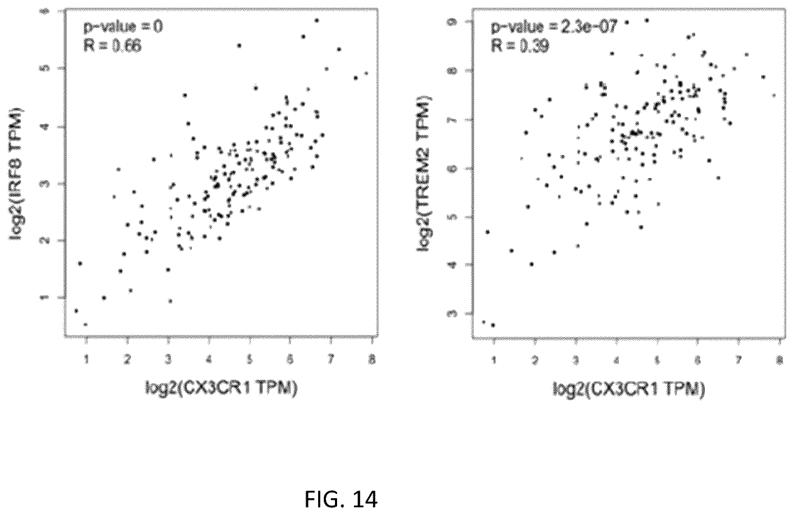

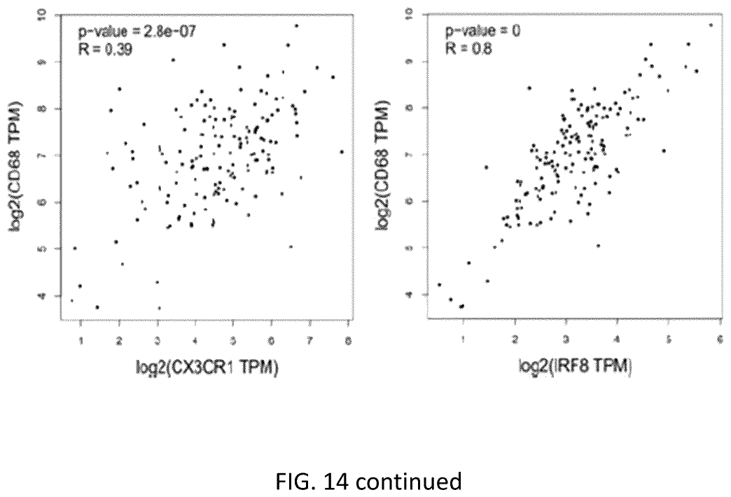

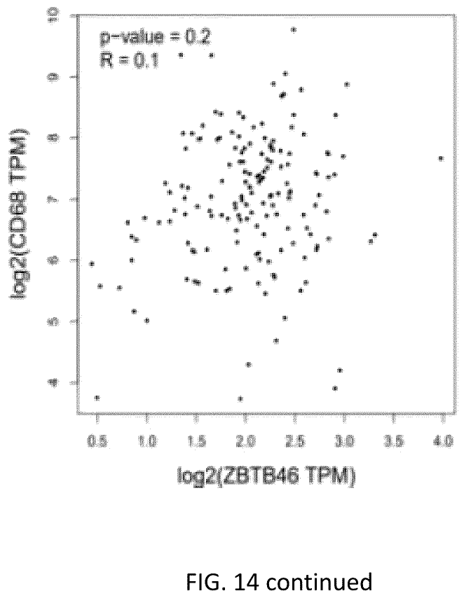

Within these primary tumors, key innate immunity pathways, specifically those involved in dendritic cell (DC) differentiation (HR 0.006; 95% CI, 0-0.167; P=0.0026) and activated microglia migration (HR 0.003; 95% CI, 0-0.114; P=0.0017), were strongly linked to improved patient survival (FIG. 4a). These pathways are essential for the activation and bridging of the innate and adaptive immune systems. Expression of 3 genes CX3CR1, TREM2, and IRF8—central regulators in these pathways and functional markers for microglia/macrophages and DCs—was highly consistent within this network and showed notable concordance with the macrophage/microglial marker CD68 (FIG. 14) and later validated through multiplex IHC analysis (FIG. 4b). Despite the evident connection of these pathways with efficacy outcomes—be it negative or positive—there were no observable differences between maximal resection or biopsy-only tumors that could account for the varied treatment responses (FIG. 4c). These findings suggest a hypothesis: bulky, biopsy-only tumors might present a unique opportunity for TTFields to engage the innate immune system, potentially facilitating an in-situ vaccination effect. This could transform the typically unresponsive T cell environment in GBM's “cold” TME—an effect that may not be as effective post-maximal resection.

v. Biopsy-Only Tumors were Associated with Enhanced Antigen-Specific T Cell Stimulation.

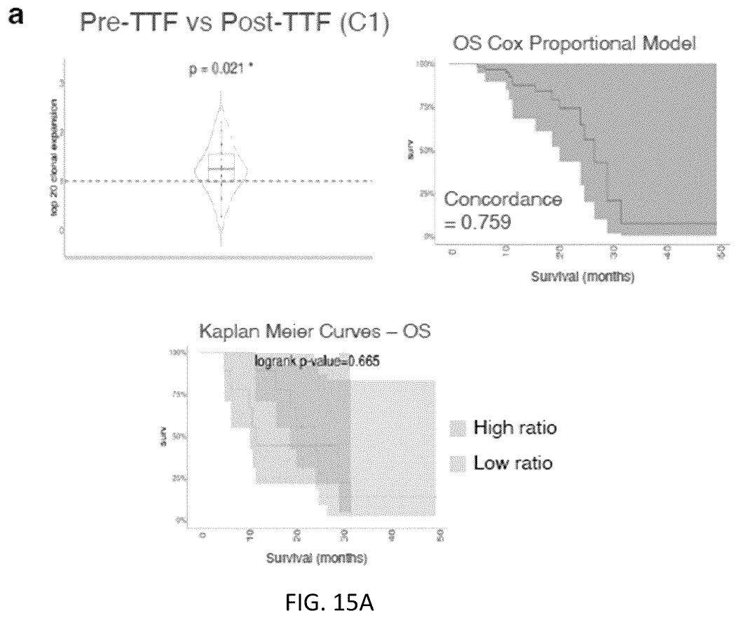

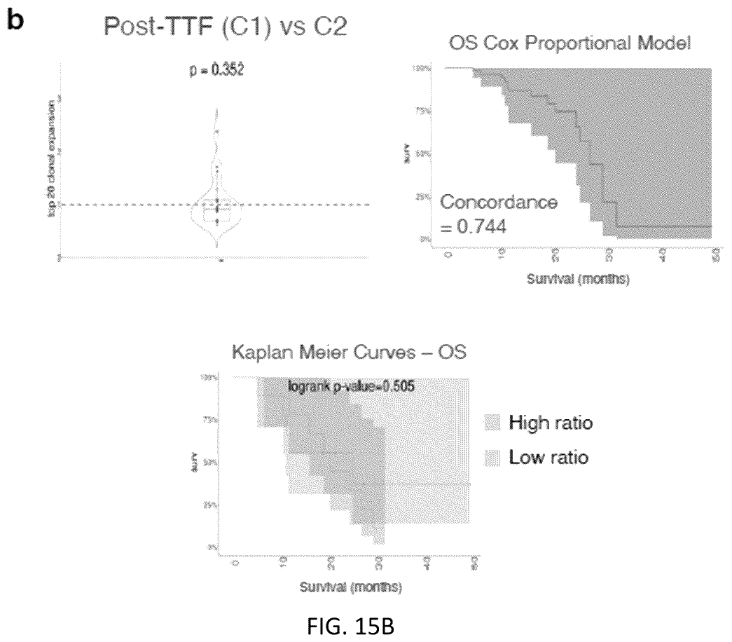

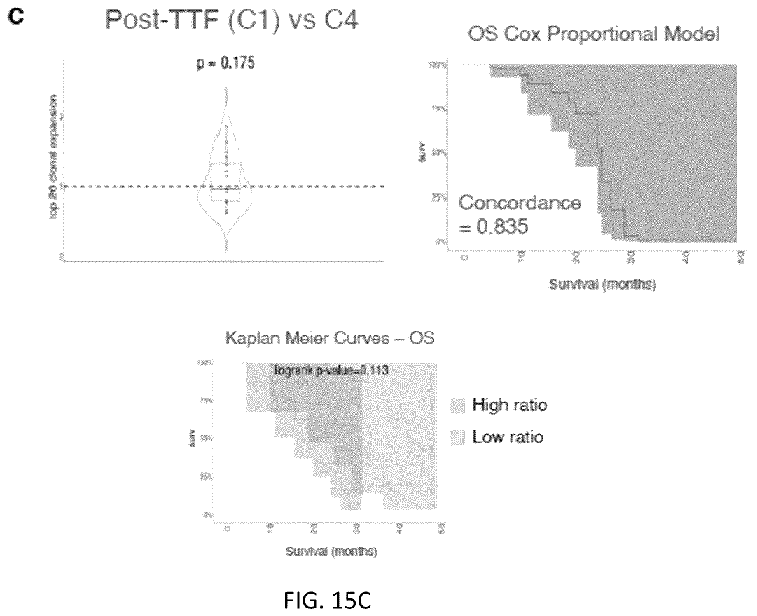

To elucidate the immunological mechanisms underlying the superior clinical outcomes in patients with biopsy-only tumors, serial PBMC samples were analyzed, due to the high risk and non-routine nature of repeated tissue sampling in CNS tumors. Moreover, peripheral adaptive immune alterations have previously been established as reliable indicators of TME dynamics in a T1IFN response in orthotopic GBM models vaccinated with TTFields-treated GBM cells with similar signal trajectory observed in TTFields-treated patients. Single-cell TCRA/B V(D)J sequencing was conducted on serial PBMCs beginning prior to TTFields treatment (Pre-TTF) and assessed TCR clonotype diversity using the Shannon diversity index. In line with earlier findings, a four-week TTFields treatment course—before adding pembrolizumab—was linked with significant TCR clonal expansion, indicative of adaptive antigen-specific immune activation (FIG. 15a, Pre-TTF vs Post-TTF or C1). However, such TCR clonal expansion was not commonly seen after commencing pembrolizumab therapy (cycles 1 to 4 or C1-4) (FIG. 15b-c). While TCRB clonal expansion showed high concordance with outcome metrics in the same multivariate Cox Proportional HR model, accounting for other variables negated any correlation with survival between groups with high versus low TCRB clonal expansion (FIG. 15, Kaplan Meier curves). This finding implies that while the initial immune activation by TTFields might be critical, its subsequent conversion to effective anti-tumor immunity likely requires synergistic action with pembrolizumab.

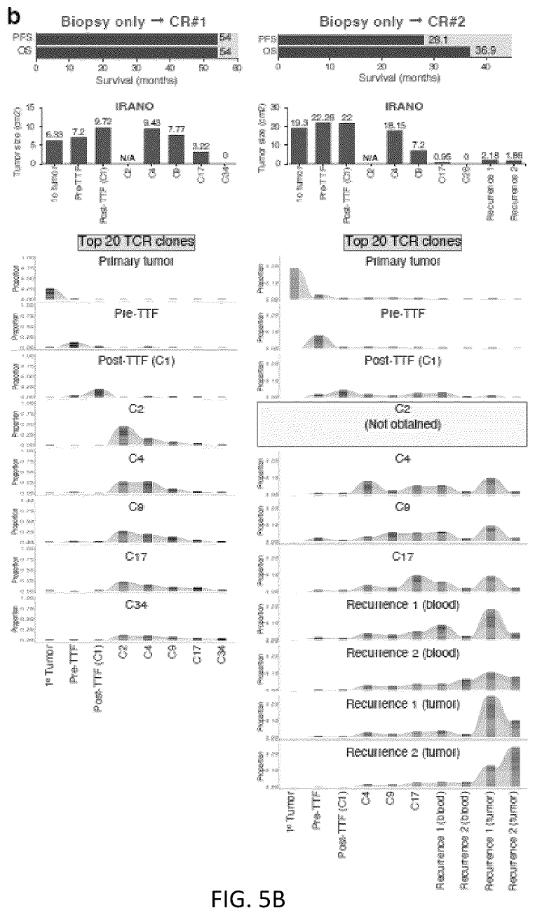

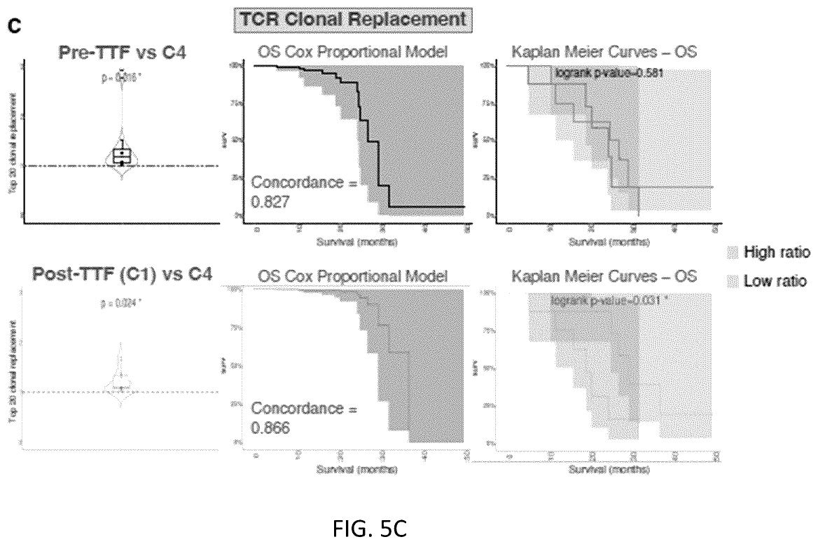

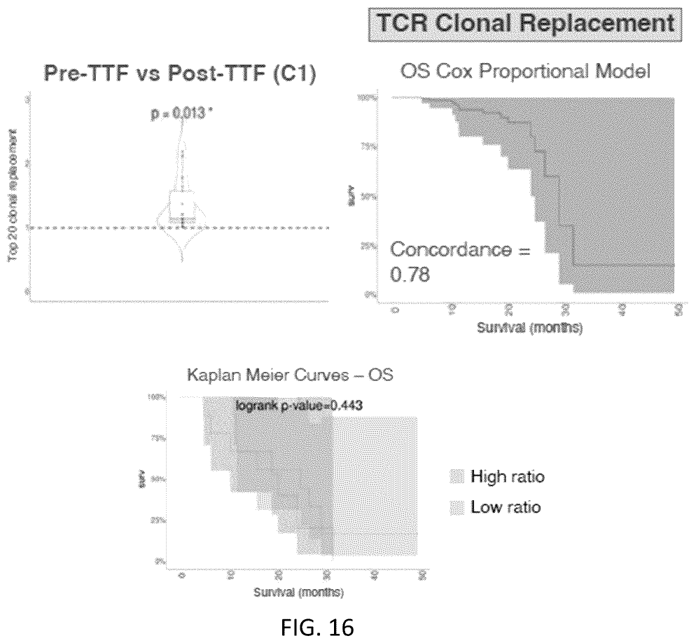

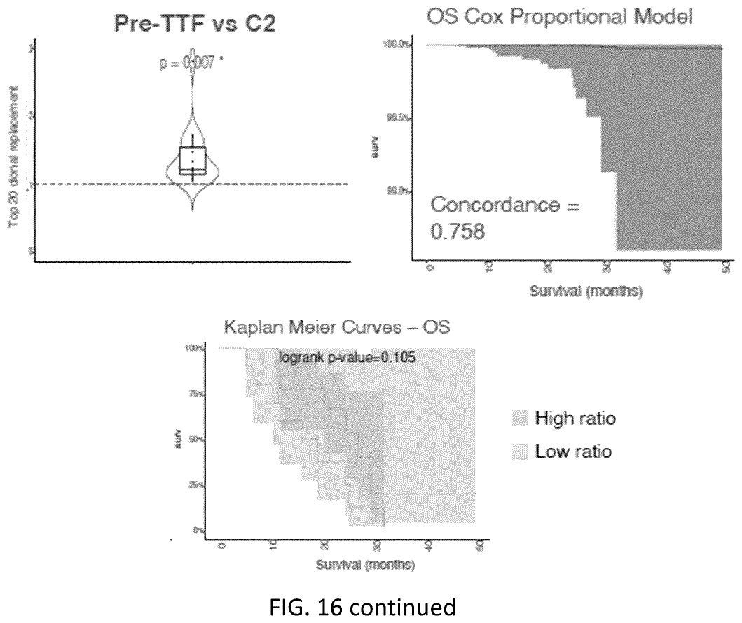

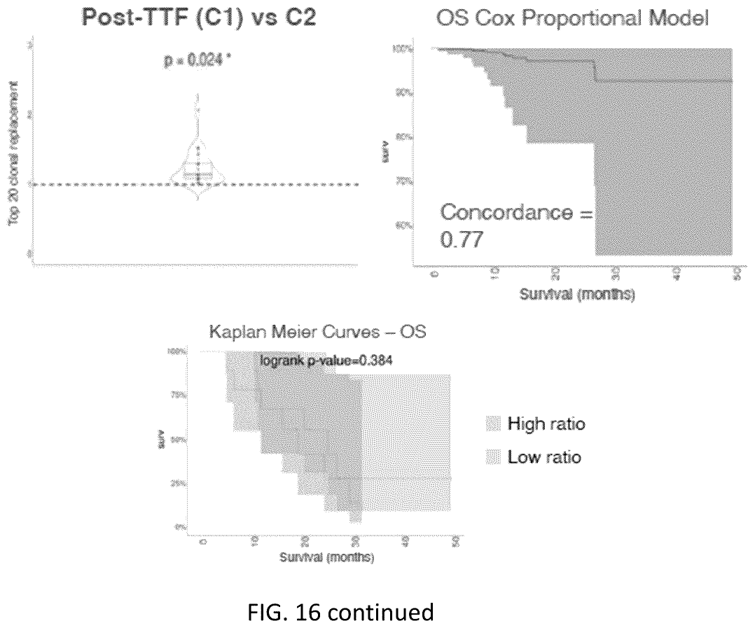

Given the role of TTFields in providing an in-situ immunizing framework for GBM, continuous TTFields application on biopsy-only tumors could catalyze the recurrent release of tumor-associated antigens (TAA), thereby facilitating the adaptive turnover of T cell clones, resulting in the expansion of new T cell clones to target emergent TAAs, effectively replacing preceding expanded clones. The phenomenon of TCR clonal replacement is crucial for the immune response's adaptability to neoantigen variation within the TME and is particularly pertinent in the context of ICI therapies. TCR clonal replacement was quantified as the ratio of the prevalence of dominant clones at a given time point to that of the previously dominant clones that had been supplanted. TCR clonal replacement profiles of 2 representative wtIDH GBM patients with maximal resection, who experienced rapid disease progression and reduced survival (PD #1 and PD #2) (FIG. 5a), in contrast to two representative responders with biopsy-only GBM who achieved complete responses and extended survival despite significant tumor burden (CR #1 and CR #2) (FIG. 5b). Analysis of the abundance of TCRB clones, ranging from the top 10 to 100 (with the top 20 depicted), and tracked over time revealed that clonal replacement was discernible by cycle 4 (C4) of pembrolizumab treatment. This replacement was markedly more apparent in patients with biopsy-only tumors relative to those with maximal resection. Notably, after correcting for other covariates, successful clonal turnover from C1 to C4, corresponding to the initial 2 months of pembrolizumab therapy, rather than from Pre-TTF to C4, was specifically predictive of a favorable therapeutic response and prolonged survival (FIG. 5c and FIG. 16), thereby implicating that while TTFields may initiate an antigen-specific immune response, pembrolizumab was essential in enhancing the adaptability of the TTFields-induced, antigen-specific immunity to the evolving TME and TAA release.

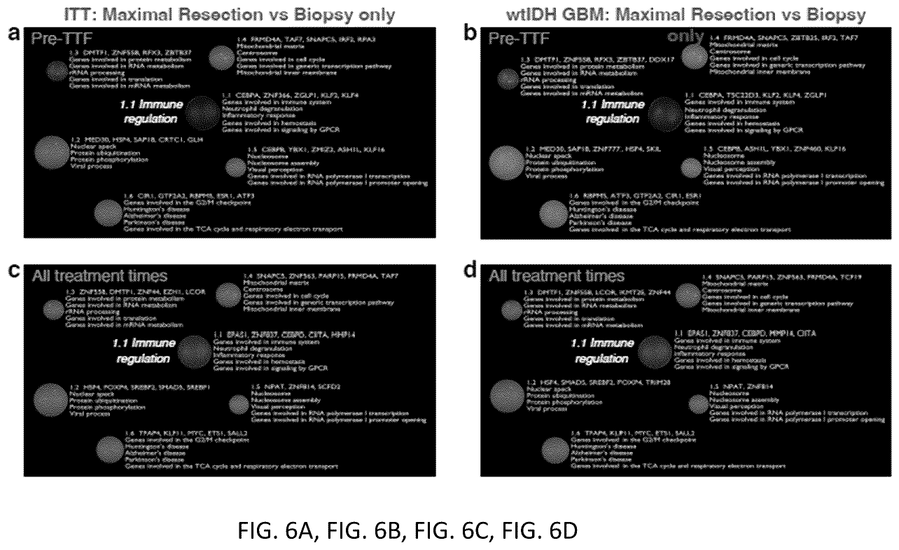

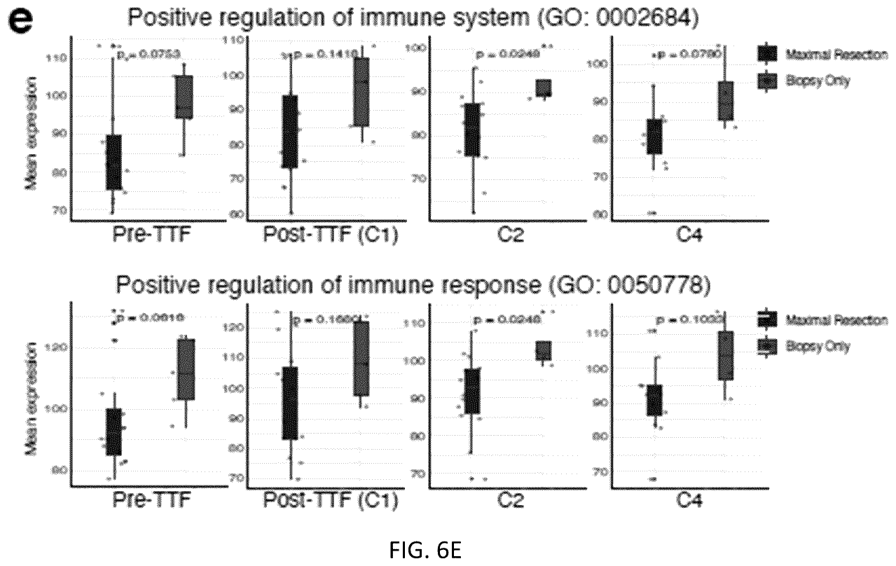

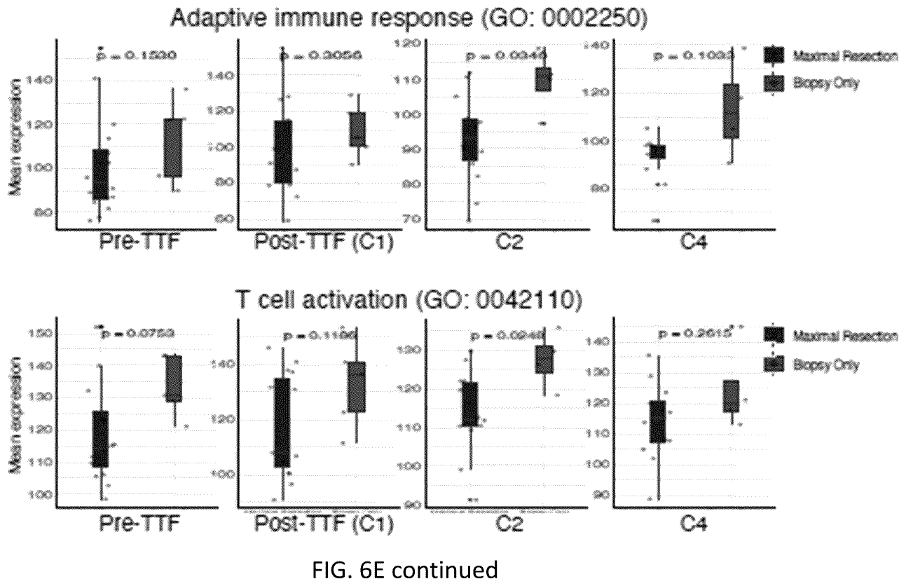

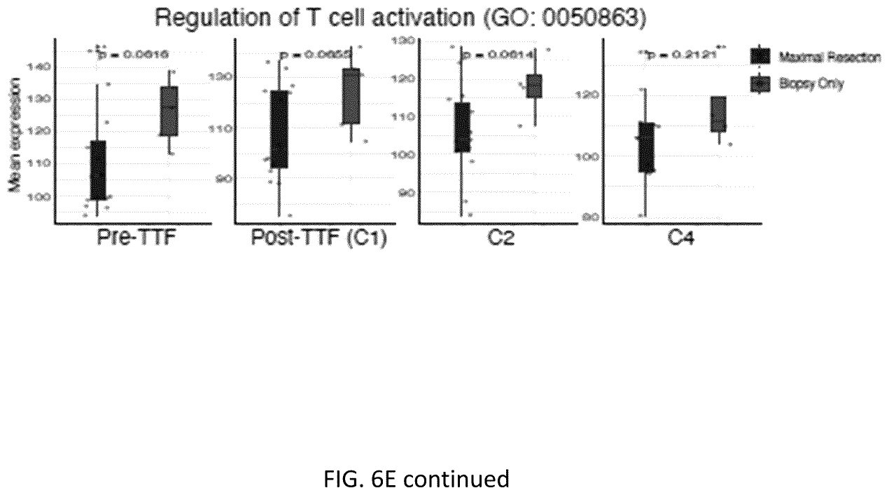

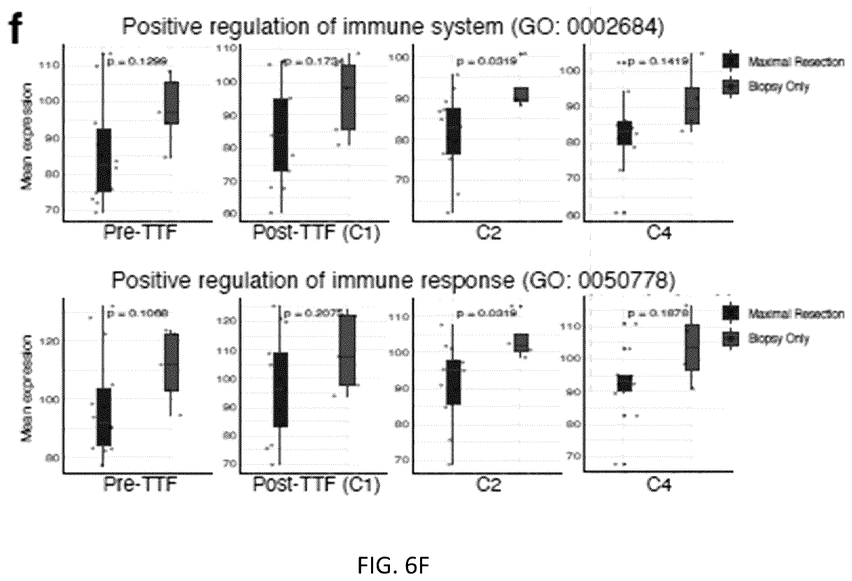

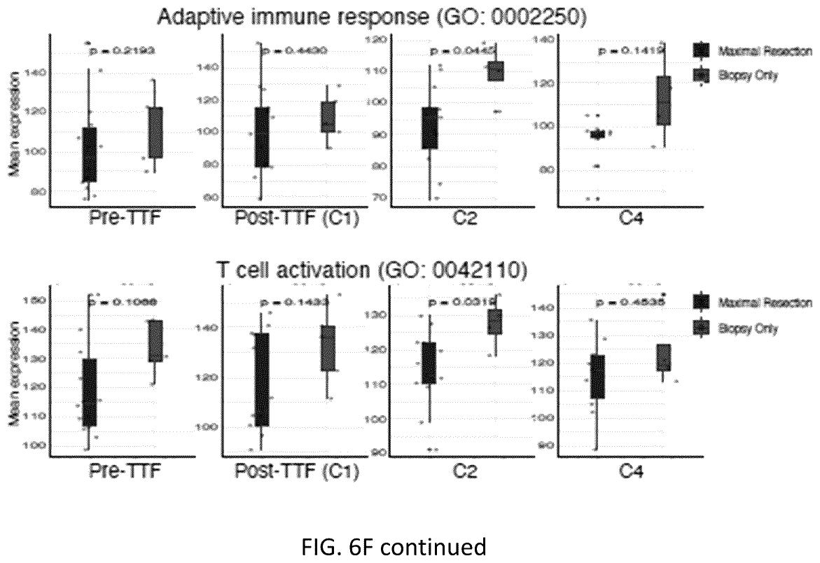

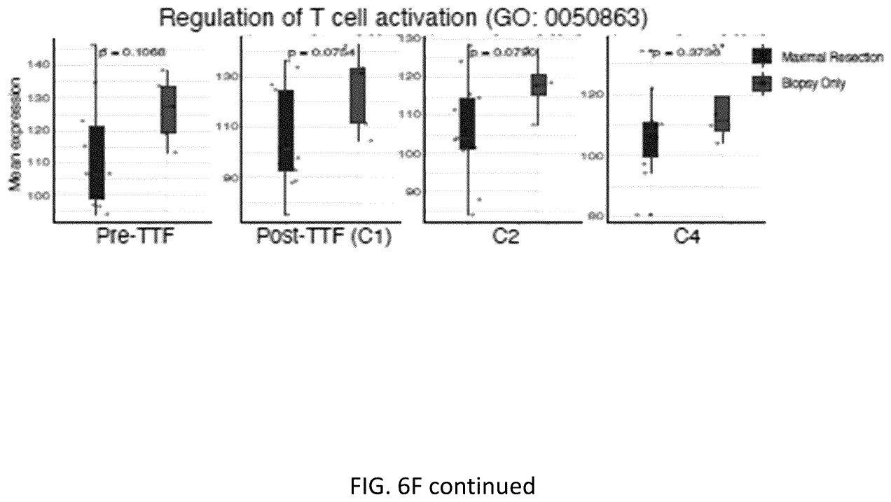

The majority of dominant TCR clones present in primary tumors were not detected in the PBMCs at the Pre-TTF timepoint, particularly in patients with biopsy-only tumors (FIG. 5a-b), suggesting that the pronounced TCR clonal replacement observed in these patients is less likely attributable to the selection and expansion of pre-existing clones that might have been induced by the prior chemoradiation. Instead, it suggests a mechanism akin to an in situ vaccination effect from TTFields, which preferentially expands novel T cell clones. To rule out the influence of prior treatments and to decode the gene networks affected by TTFields and the combination of TTFields with pembrolizumab, T cells were isolated from PBMCs using CD3-negative selection and subjected to bulk deep RNA-seq. Analysis was conducted using GeneRep/nSCORE, a gene network-based machine learning algorithm refined for precision medicine, which is augmented by an automated 3D network visualization pipeline, to probe comprehensive gene expression alterations and pathway deviations subsequent to therapy. Large signaling complexes that govern multiple facets of T cell biology, including a pivotal immune regulatory hub, were identified and charted. At the Pre-TTF timepoint—occurring no less than four weeks post standard chemoradiation—the immune regulatory hub appeared downregulated in biopsy-only patients relative to those with maximal resection (FIG. 6a-b). However, this hub was subsequently upregulated in biopsy-only patients (FIG. 6c-d). These findings endorse the notion that increased T cell activation and the likely heightened rates of TCR clonal turnover in biopsy-only tumors are direct consequences of TTFields and subsequent combination therapy with pembrolizumab, rather than lingering effects of the antecedent chemoradiation regimen. Indeed, within 5 key T cell regulatory pathways of the immune hub, ranging from broad immune system regulation to those more narrowly confined to T cell activity as classified by the Gene Ontology collection, biopsy-only tumors exhibited a markedly elevated activation status. However, this activation was not evident at the Pre-TTF juncture but became significant at later stages, notably at C2 following pembrolizumab administration in both the ITT and wtIDH GBM only populations (FIG. 6e-f).

In summary, TTFields instigate an adaptive immune response that is further enhanced by the anti-PD-1 immunotherapy pembrolizumab. This combination potentiates the immune system's capacity to adapt and mount an effective anti-tumor response, particularly in patients with non-resectable, bulky tumors. The findings underscore the synergistic in situ vaccination effect elicited by the concurrent application of TTFields and pembrolizumab.

vi. TTFields Combined with Pembrolizumab Enhanced Central Memory T Cell Development in Representative Responders with Biopsy-Only Tumors.

TCR clonal replacement manifested rapidly early in the treatment regimen among patients with biopsy-only tumors, with the most expanded clones stabilizing after the fourth cycle (C4) (FIG. 5b), suggesting a potential equilibrium in TCR clonotype selection. To delve into the nuances of TCR clonal evolution that may delineate peripheral immune shifts between responders and non-responders, the expansion and activation of individual T cells and TCR clonotypes were profiled in response to TTFields and pembrolizumab, using single-cell RNA-seq of PBMCs and TCR sequences from the same 2 biopsy-only responders CR #1 and CR #2 and 2 maximal resection non-responders PD #1 and PD #2. Single PBMCs were analyzed using the graph-based Seurat R package for clustering, supplemented with UMAP for dimensionality reduction.

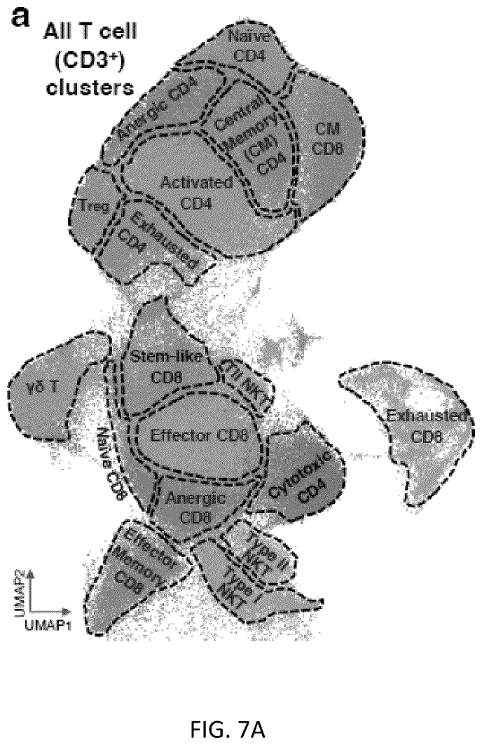

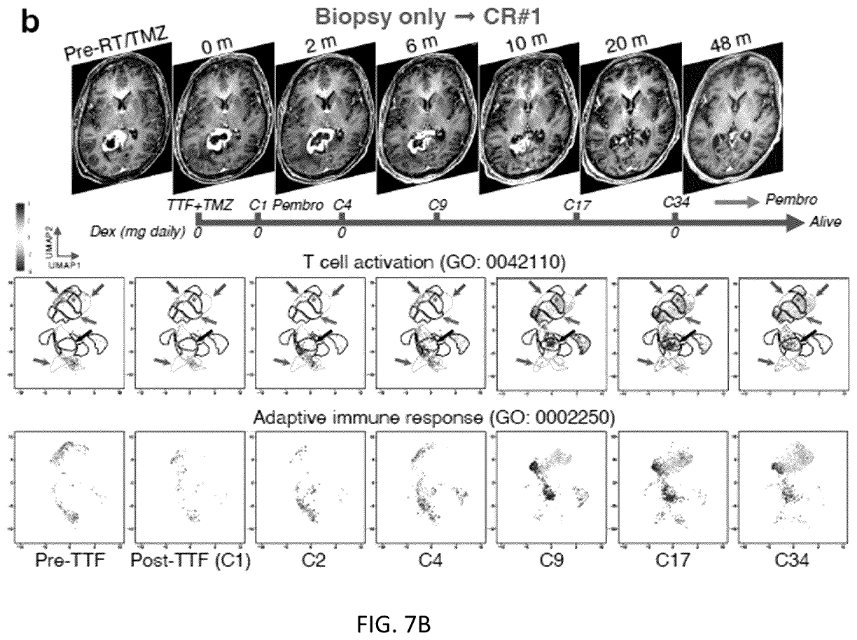

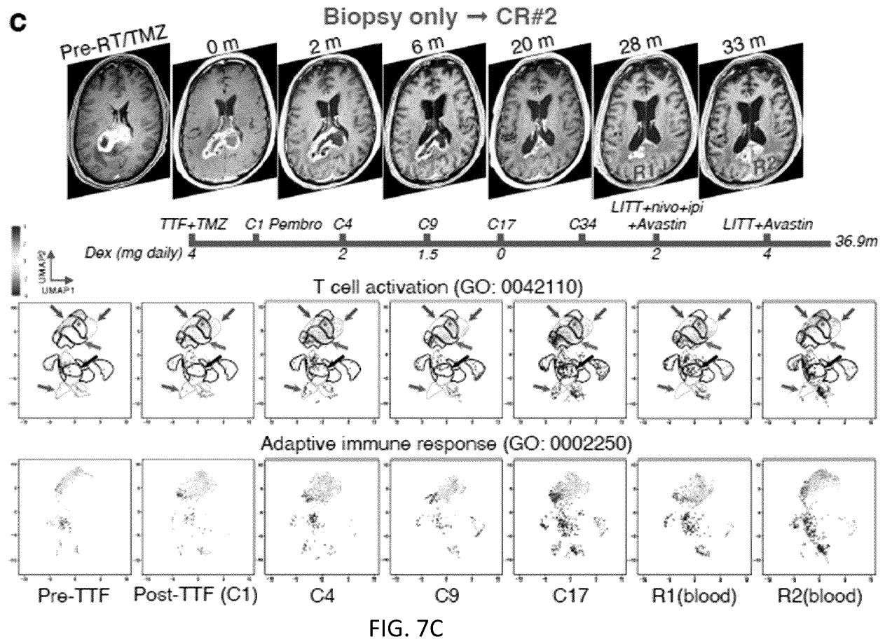

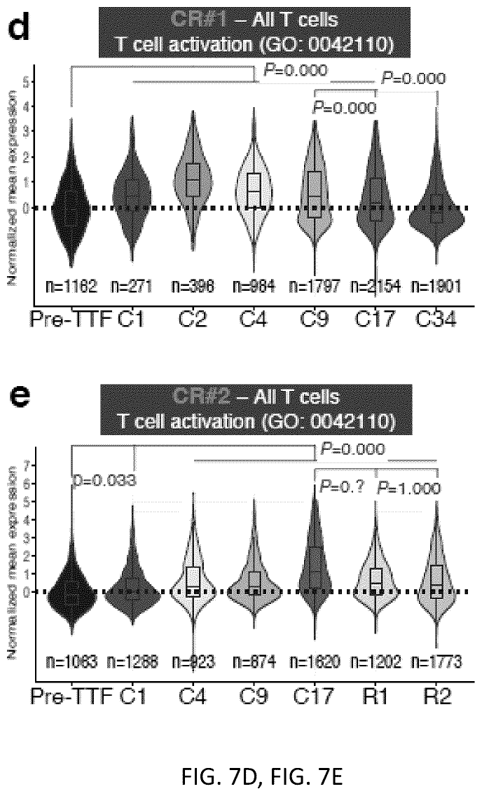

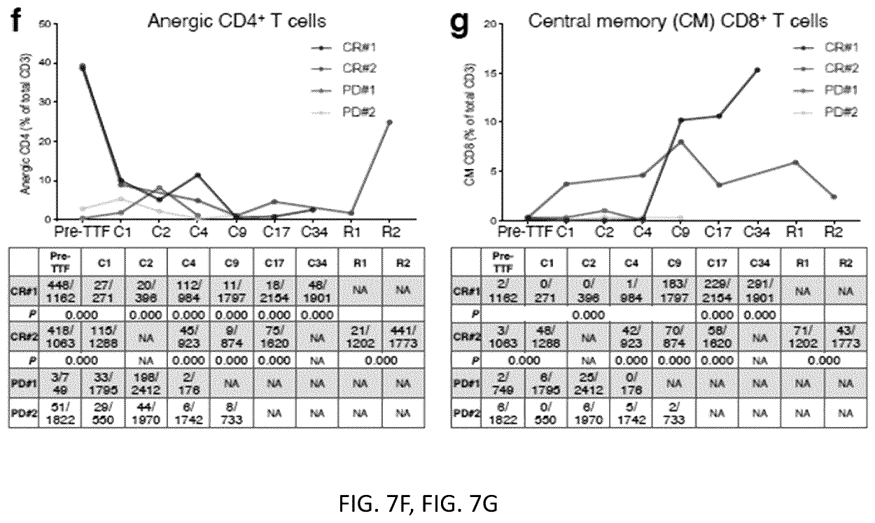

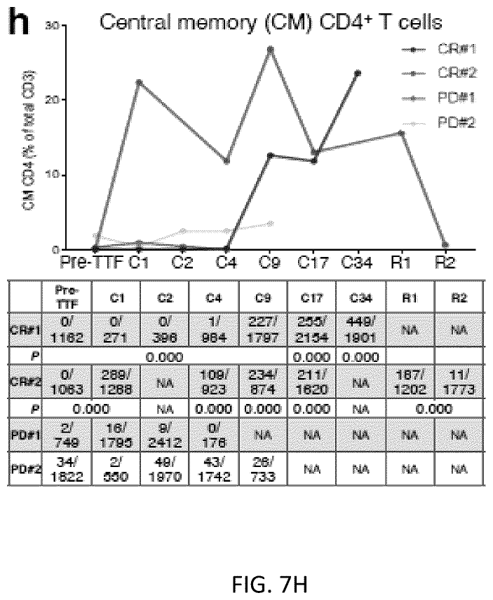

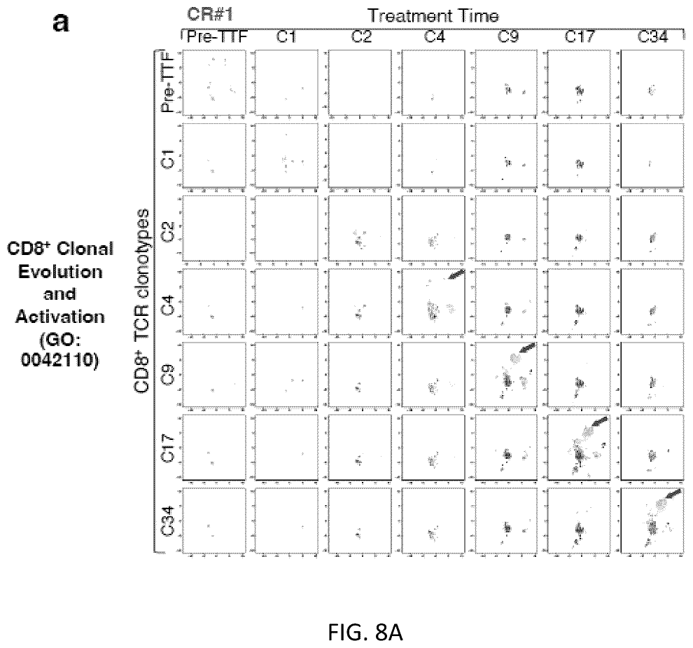

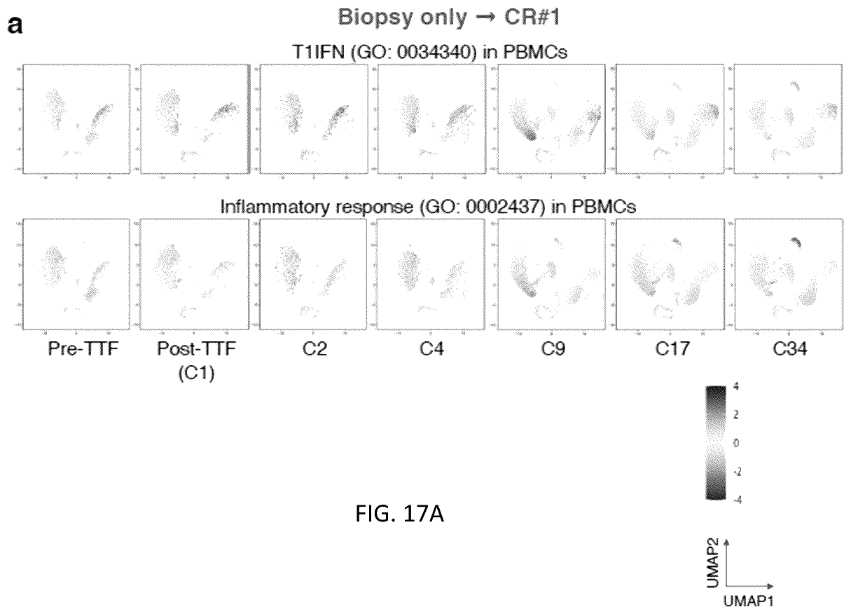

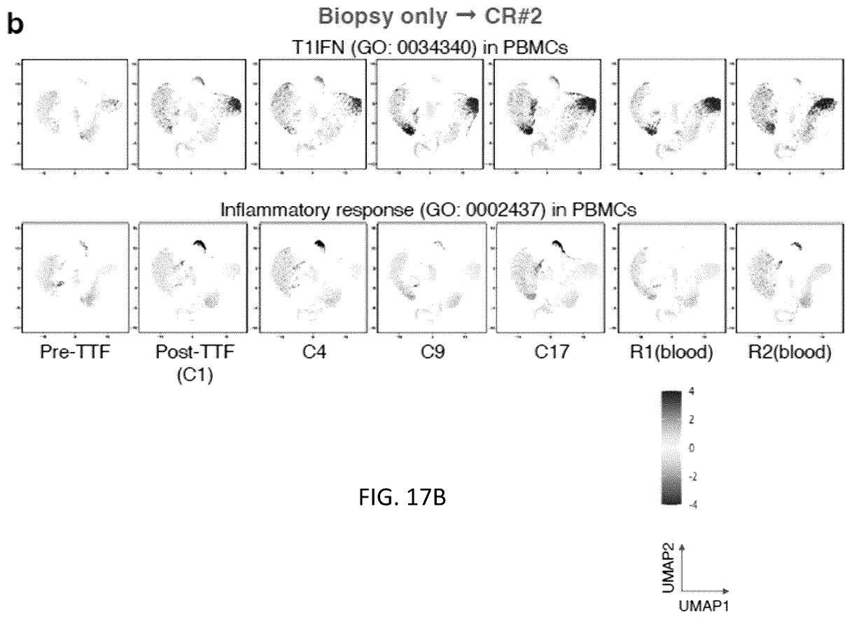

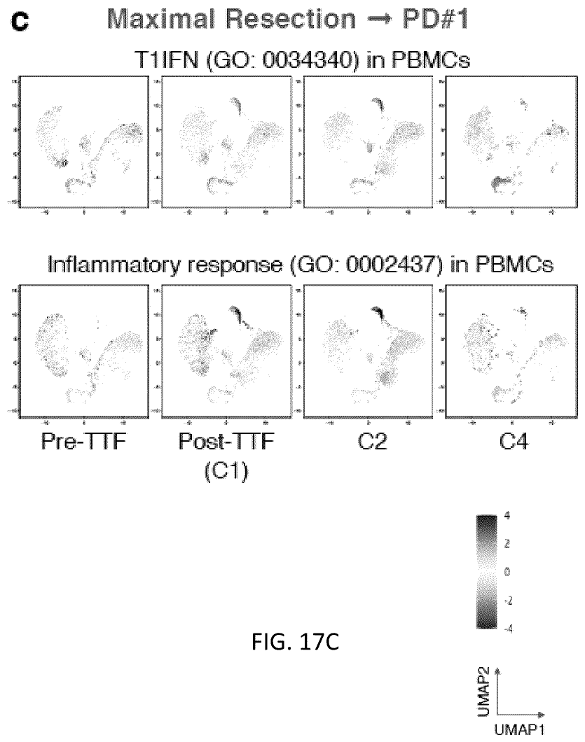

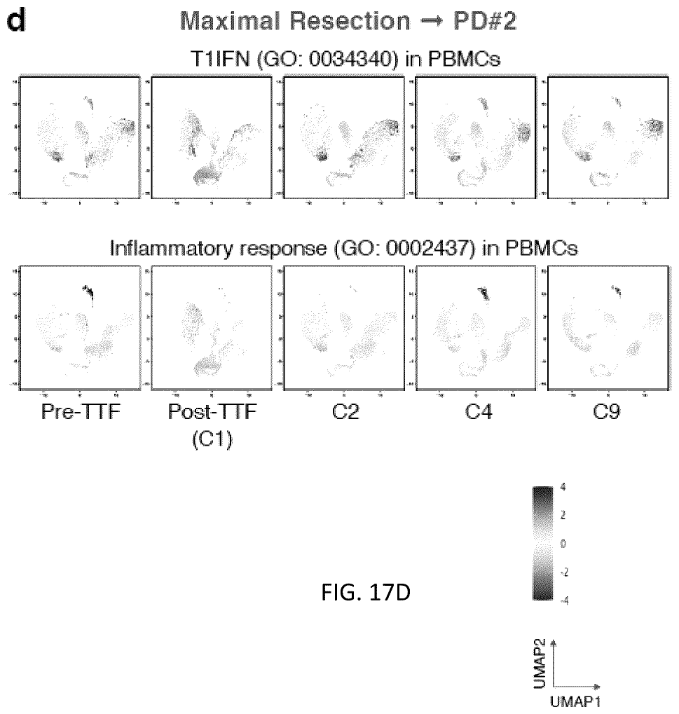

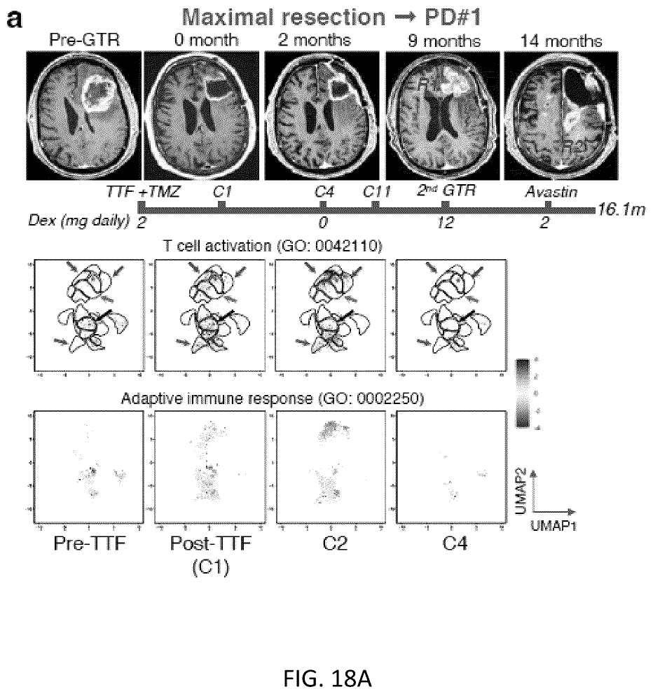

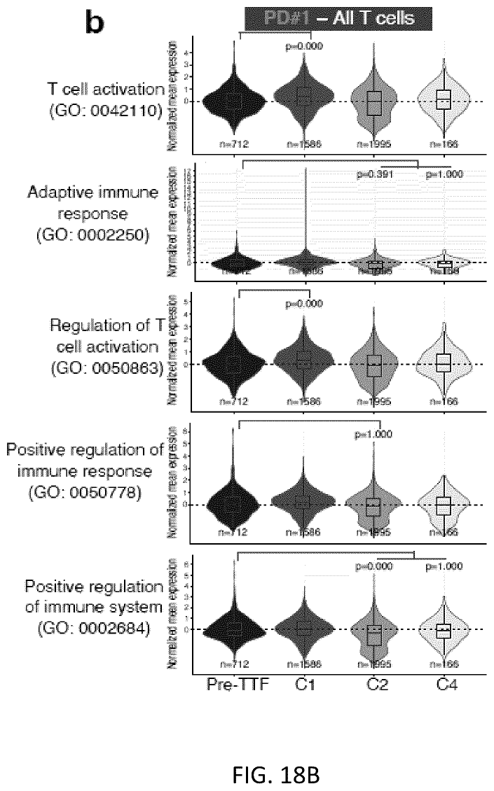

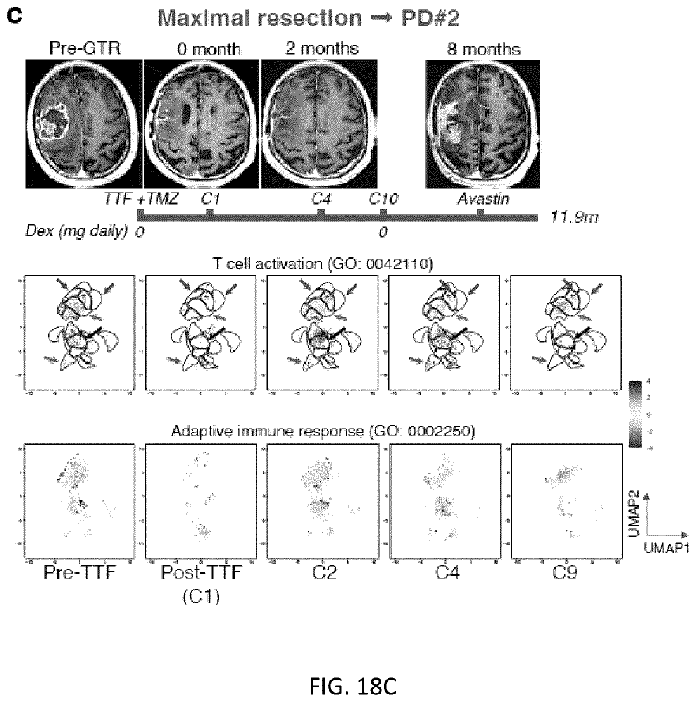

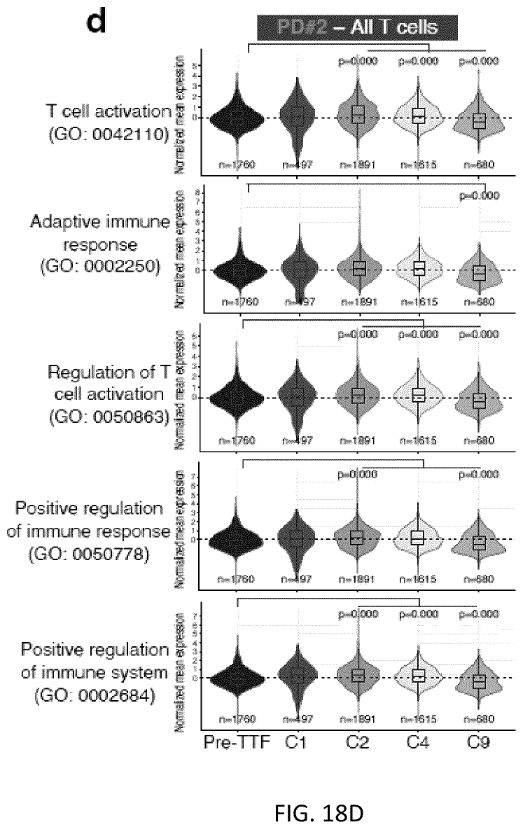

Consistent with the expected tumor immunizing effect of TTFields through cGAS/STING and AIM2/Caspase1 activation, it was observed that TTFields promoted a T1IFN-driven immune response in PBMCs, as opposed to a non-T1IFN inflammatory trajectory, particularly in CR #1 and CR #2 patients, unlike the trajectory in PD #1 and PD #2 patients (FIG. 17). To map how the activation signals emanated from T1IFN-driven alterations to T cell subsets, T cells were isolated based on CD3 expression and applied a gene set indicative of T cell identity and function to delineate CD4⁺ and CD8⁺ clusters. Single-cell evaluations of mean expression levels were conducted for the same 5 GO T cell signaling pathways, observing comparable patterns across all 5 cases. In the biopsy-only tumors of CR patients, anergic and naïve CD4⁺ T cell clusters were densely populated with inactive cells prior to TTFields therapy (FIG. 7b-c, purple arrow), in contrast to the sparser distribution in PD patients with maximally resected tumors (FIG. 18a, 18c). T cell anergy and senescence are prominent dysfunctions within T cell populations in GBM patients, attributable to the systemic immunosuppressive state associated with the disease. Variability was noted in the occupancy of naïve and anergic CD8⁺ T cell clusters in Pre-TTF samples across all 4 patients. Notably, the compartment for activated effector CD8⁺ T cells was virtually unoccupied in CR patients (FIG. 7b-c, black arrow), whereas it was crowded with activated cells in non-responders (FIG. 18a, 18c). These findings align with previous global expression analyses of the TME and peripheral T cells (FIG. 6), indicating that the adaptive immune system in patients with biopsy-only tumors was relatively suppressed post-standard chemoradiation, likely due to the large tumor mass, in contrast with patients who had maximal tumor resection. Nevertheless, the large residual tumors in biopsy-only patients might also create optimal conditions for enhanced in situ immune activation by the treatment regimen.

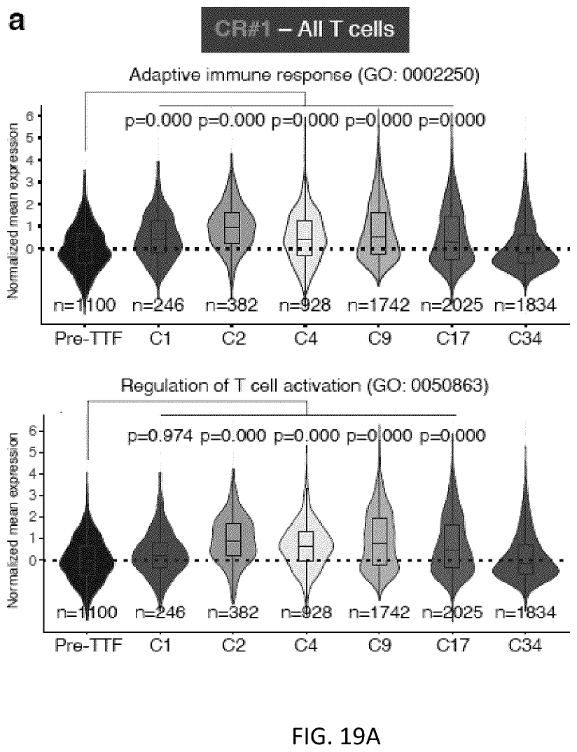

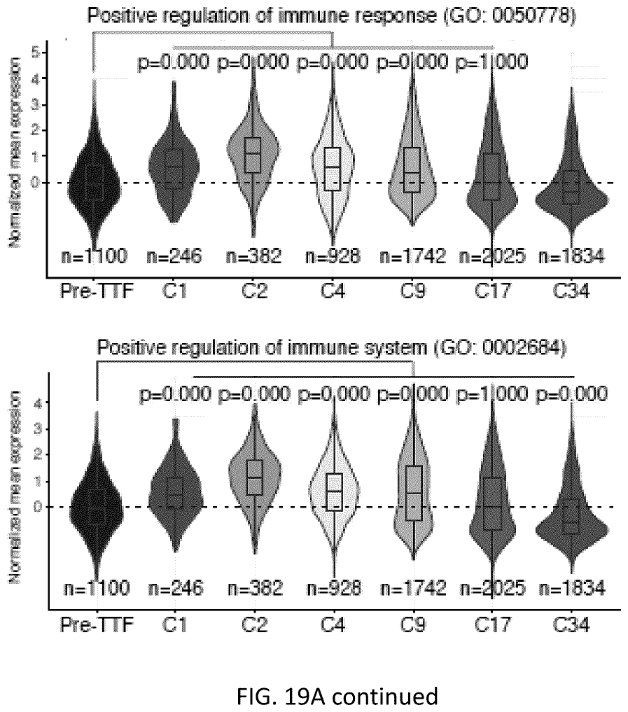

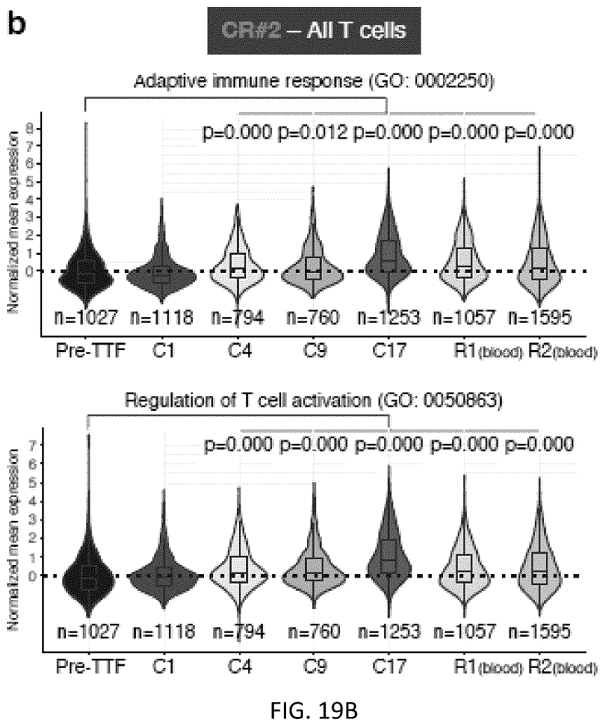

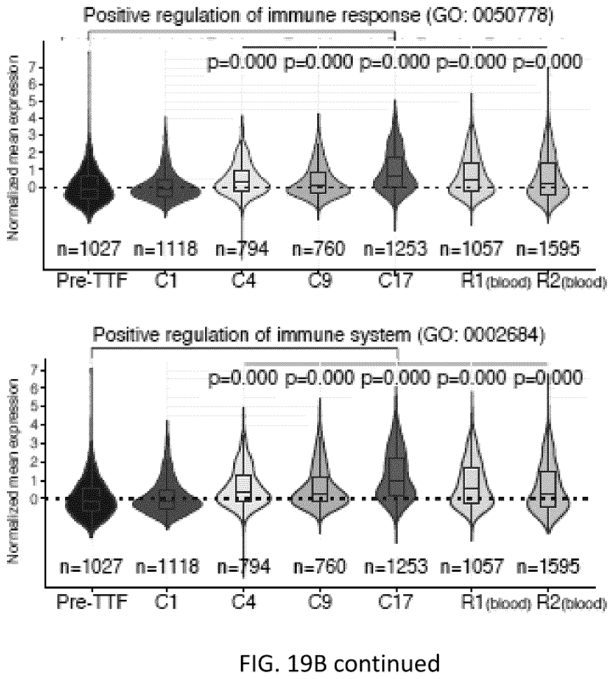

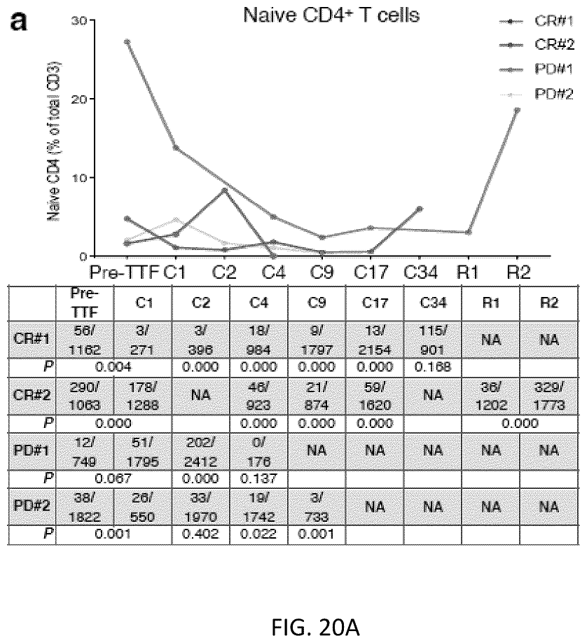

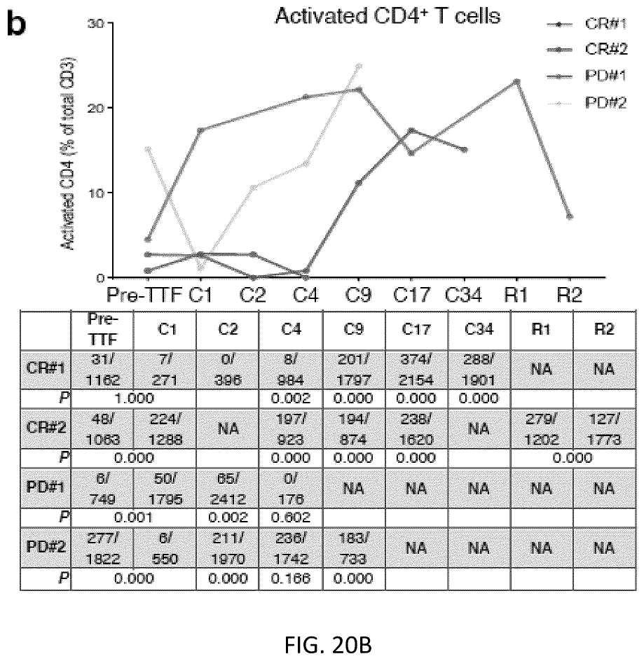

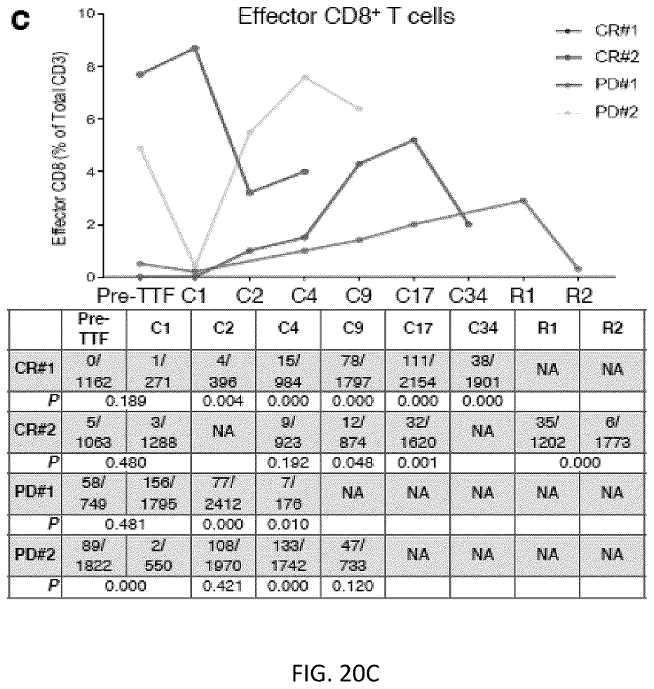

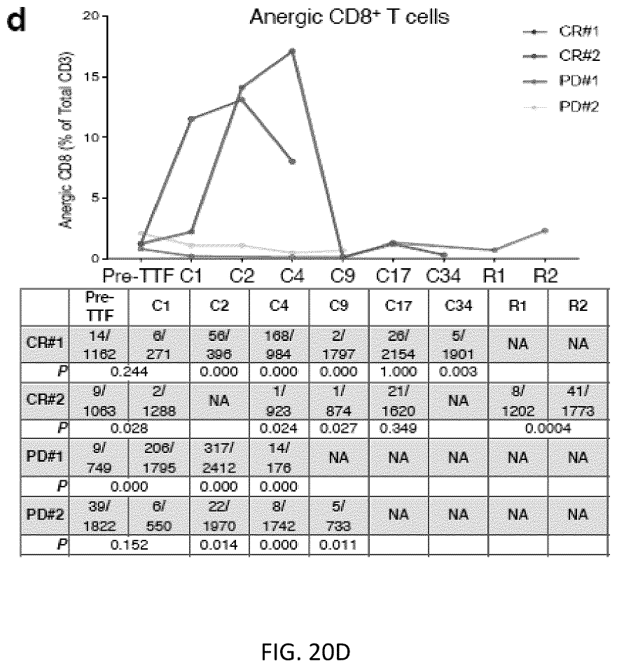

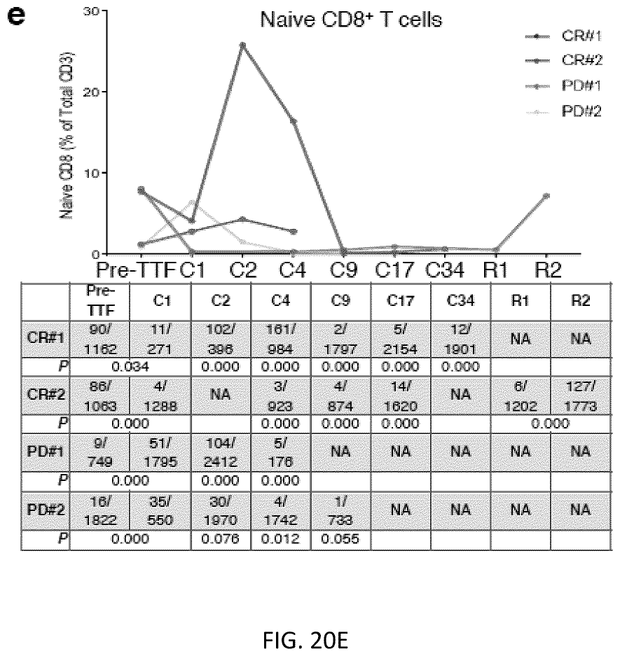

Indeed, following the initiation of TTFields and pembrolizumab, there was a significant activation of all 5 GO T cell activation pathways, specifically in CR (FIG. 7d-e and FIG. 19) but not PD (FIG. 18b, 7d) patients. This activation was concurrent with a rapid reduction of anergic and naïve T cell populations and the ensuing emergence of activated CD4⁺ (green arrow), effector CD8⁺ (black arrow), effector memory CD8⁺ (blue arrow), and eventually central memory (CM) CD4⁺ (red asterisk) and CM CD8⁺ (red arrow) T cells (FIG. 7b-c-enumerated in FIG. 7f-h and FIG. 20). Importantly, this immunological shift was apparent by C9 of pembrolizumab, coinciding with the initial observable tumor shrinkage (see also FIG. 5). In stark contrast, while PD patients also exhibited formation of activated CD4⁺ and effector CD8+ T cells, the response was inconsistent and transient, with minimal generation of CM CD4⁺ T cells and an almost complete lack of CM CD8⁺ T cell development, highlighting a major discrepancy in the immunological response to treatment between the responder and non-responder groups (FIG. 18—enumerated in FIG. 7f-h and FIG. 20).

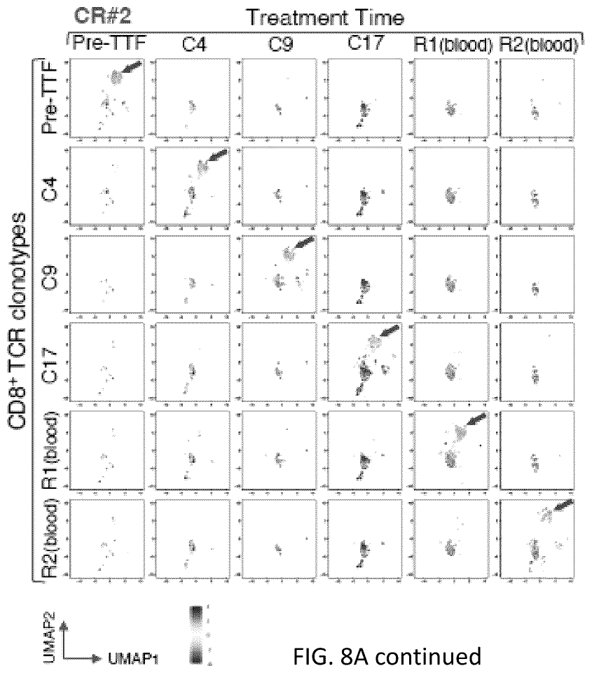

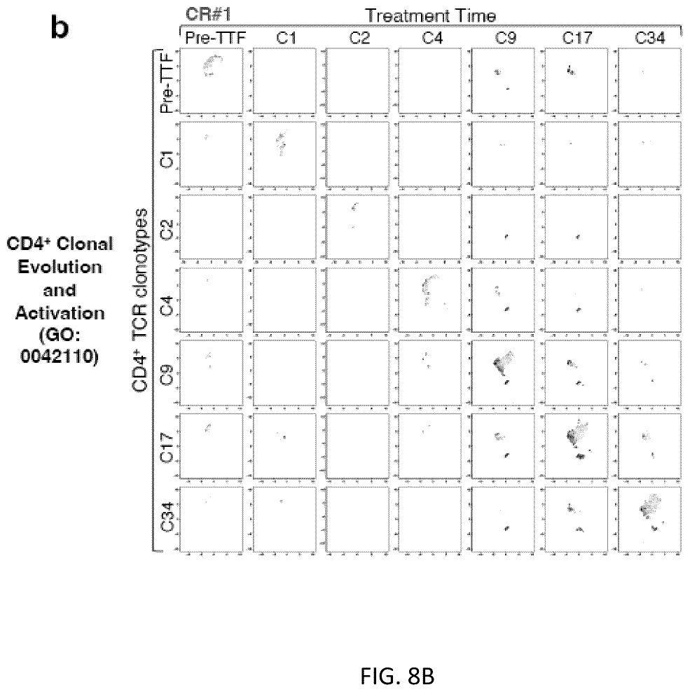

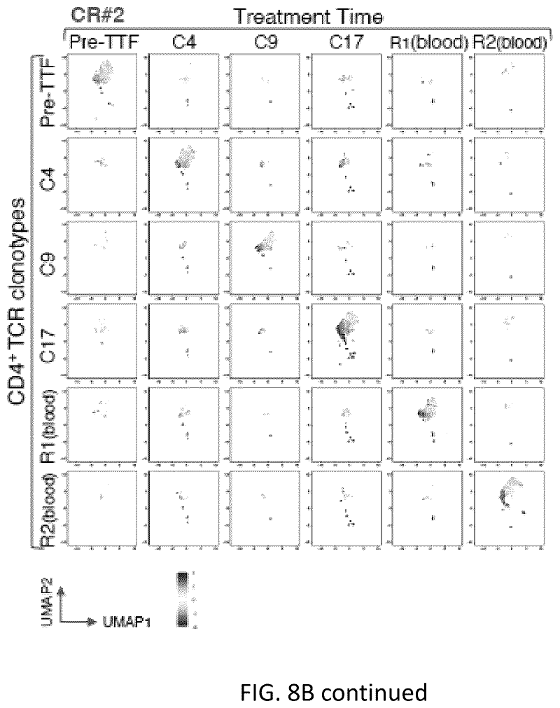

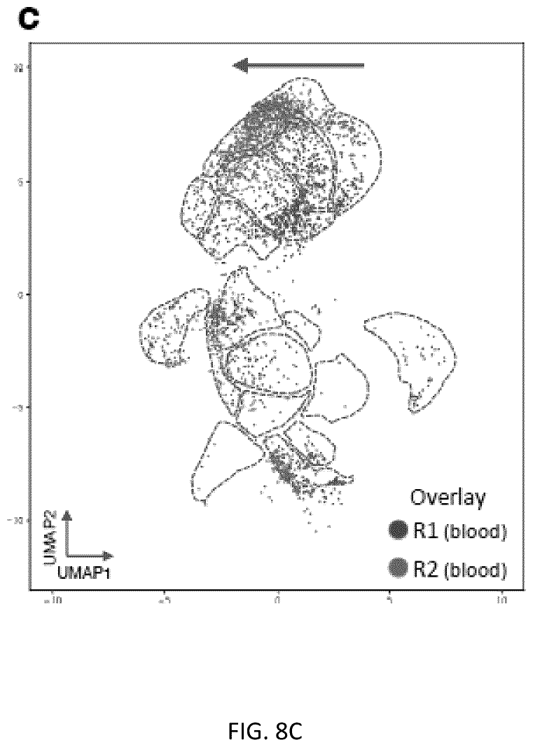

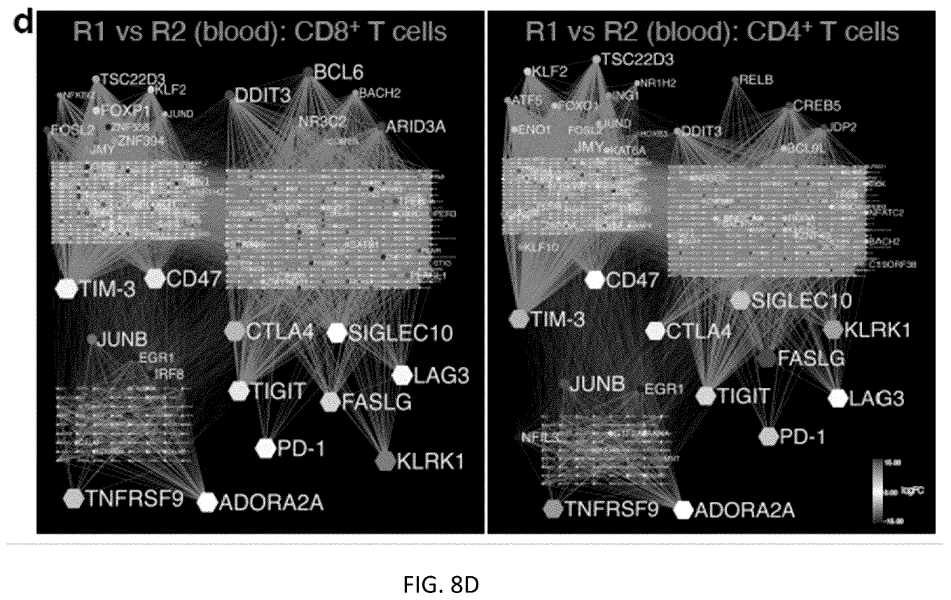

Lastly, the functional progression and activation state (GO: 0042110) of individual CD4⁺ and CD8⁺ T cell clonotypes were monitored to evaluate the dynamics of clonal turnover in patients CR #1 and CR #2. Interestingly, in both cases, numerous dominant CD8⁺ T cell clonotypes across different treatment timepoints not only persisted but expanded in their prevalence and increased in activation, diversifying into various functional subtypes instead of being entirely supplanted, with the exception observed within the CM compartment (FIG. 8a-indicated by the red arrow). Conversely, a majority of CD4⁺ T cell clonotypes, spanning numerous non-CM functional states and including quiescent CM cells, were replaced between treatment intervals by newly activated non-CM clones (FIG. 8b-CM denoted by red arrow). These findings suggest that the turnover of CD4⁺ TCR was the predominant contributor to the overall TCR clonal replacement activity in this cohort, particularly following the introduction of pembrolizumab. This underscores the pivotal role of CD4⁺ T cells and their dynamic clonal replacement in mediating treatment response, which is instrumental in the selective expansion and promotion of antigen-specific CD8⁺ effector T cells and the establishment of CM T cell populations, crucial for efficacious tumor eradication.

vii. Systemic and TME Reprogramming May Induce Resistance by Activating Alternative Immune Checkpoints.

In patient CR #2, peripheral T cell functionality was sustained at the first tumor recurrence (R1), but a comparative analysis by overlaying UMAPs suggested a shift from an activated and memory state in R1 toward a systemic immunosuppressive, anergic state in the second recurrence (R2) (FIG. 8c). Specifically, there was a marked increase in the proportions of anergic and naive CD4⁺ and CD8⁺ T cells, reverting toward baseline levels observed before TTFields (Pre-TTF) treatment (FIG. 7f and FIG. 20a, d, e). Concurrently, the fractions of activated CD4⁺, effector CD8⁺, CM CD4⁺, and CM CD8⁺ T cells substantially decreased in R2 compared to R1 (FIG. 7g-h and FIG. 20b-c). Of note, this pattern contrasts with the changes seen in patient CR #1, where an increase in naive CD4⁺ T cells did not coincide with anergy. Instead, there was a reduction in activated T cell fractions—though still above Pre-TTF levels—and a sustained presence of CM T cells, suggesting a systemic return to a quiescent yet primed memory state in the context of a durable complete response.

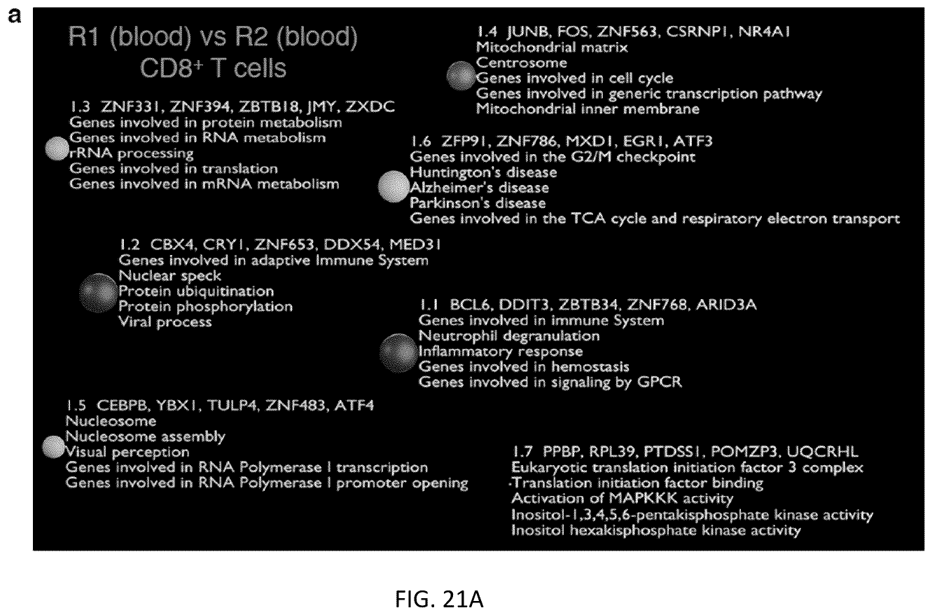

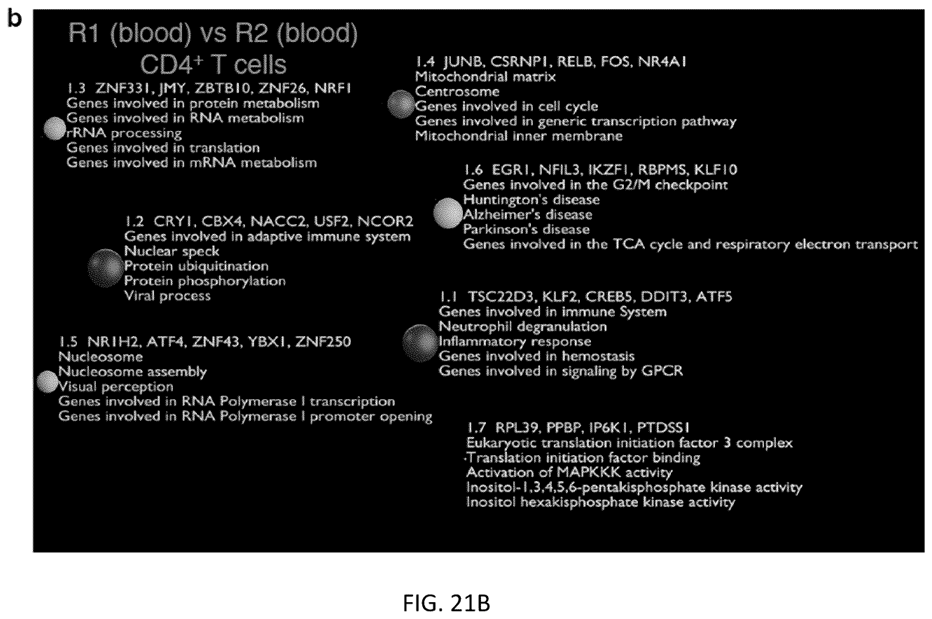

Upon analyzing the master regulatory network using GeneRep/nSCORE in R2 blood, a downregulation of major hubs involved in general and adaptive immune signaling was observed. Interestingly, the primary proliferative hub showed upregulation (FIG. 21). Between R1 and R2 blood, key negative regulators of T cell proliferation and homeostasis, such as JunB, JunD, and RelB, were downregulated, while PD-1 expression decreased, likely in response to ongoing anti-PD-1 therapy. In contrast, alternative immune checkpoints like CTLA4, TIM-3, and TIGIT saw an increase in expression. Moreover, critical TCR co-stimulatory receptors, particularly TNFRSF9 and KLRK1, were significantly elevated (FIG. 8d). These conflicting signals point to a potential but inadequate compensatory mechanism attempting to counteract the escalating systemic immunosuppression associated with tumor recurrence.

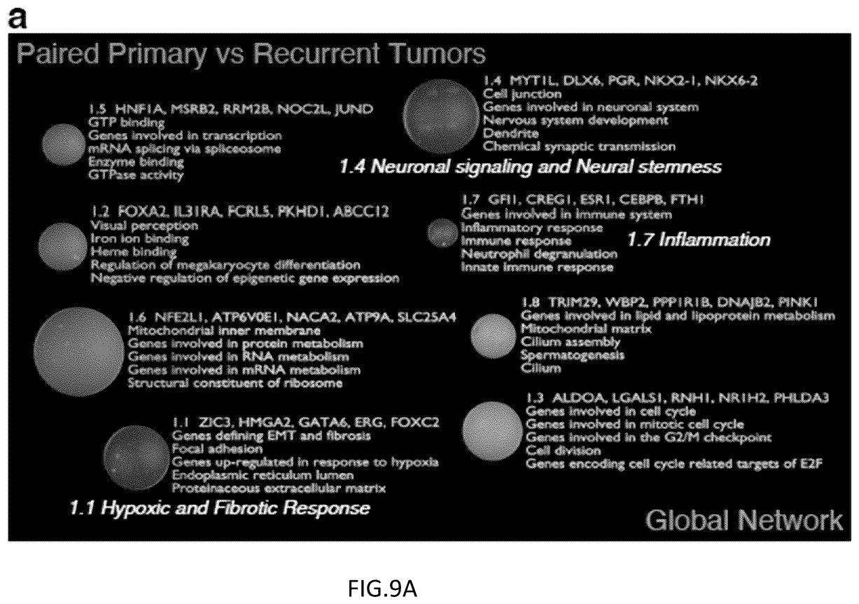

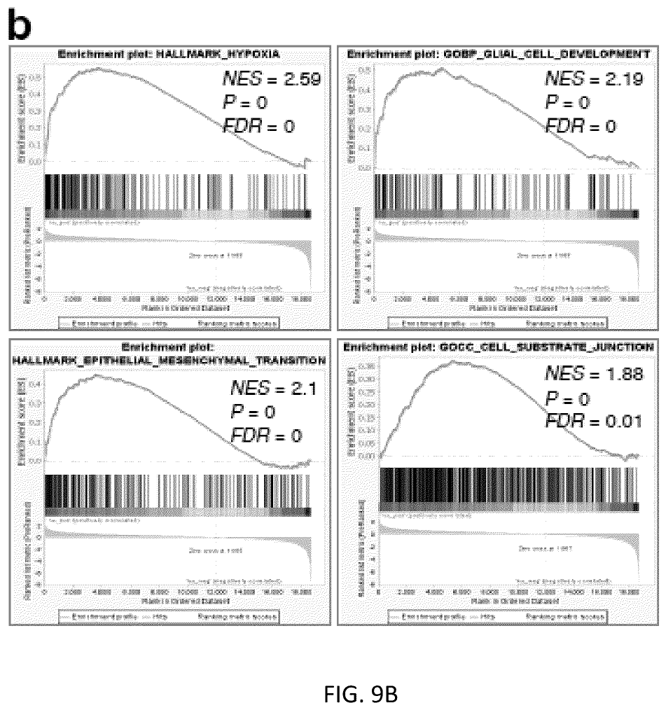

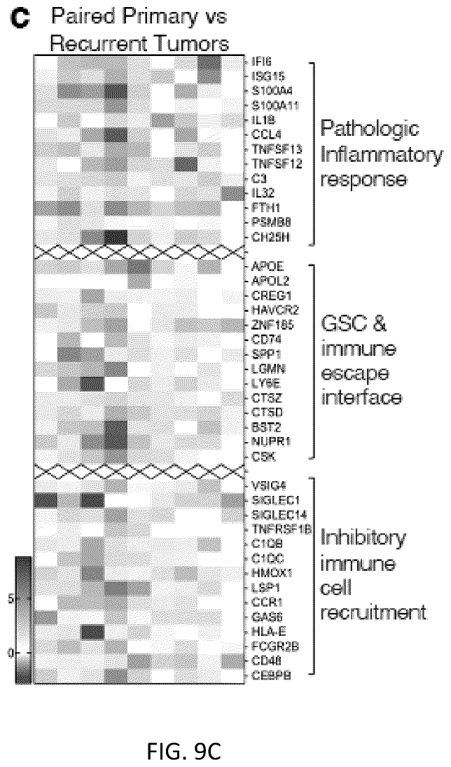

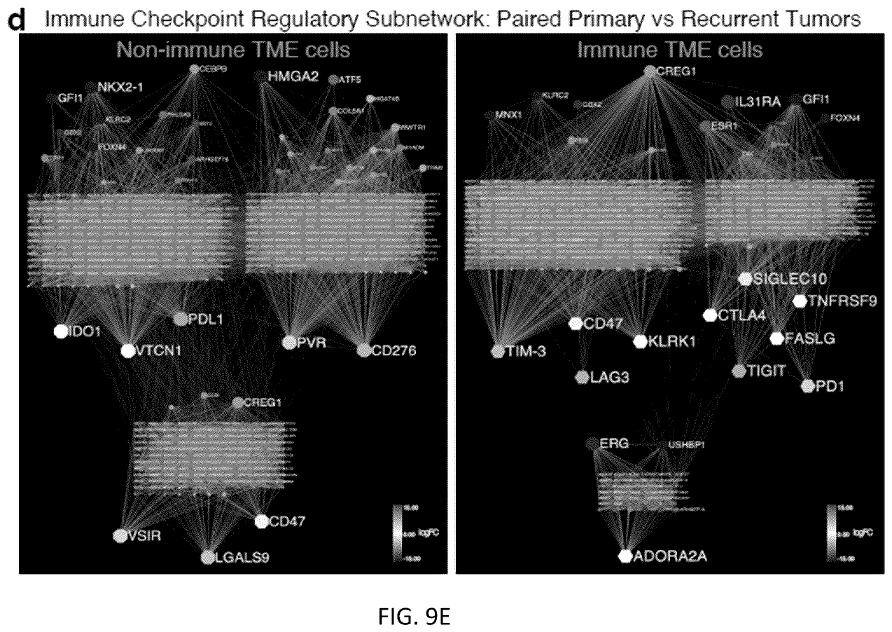

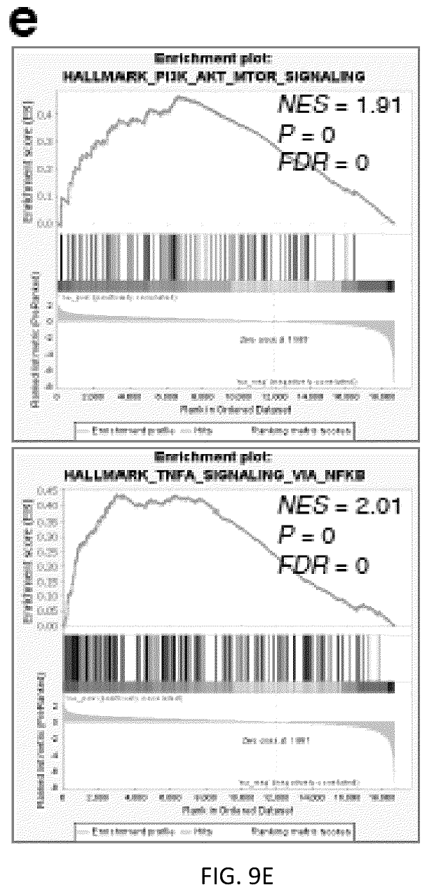

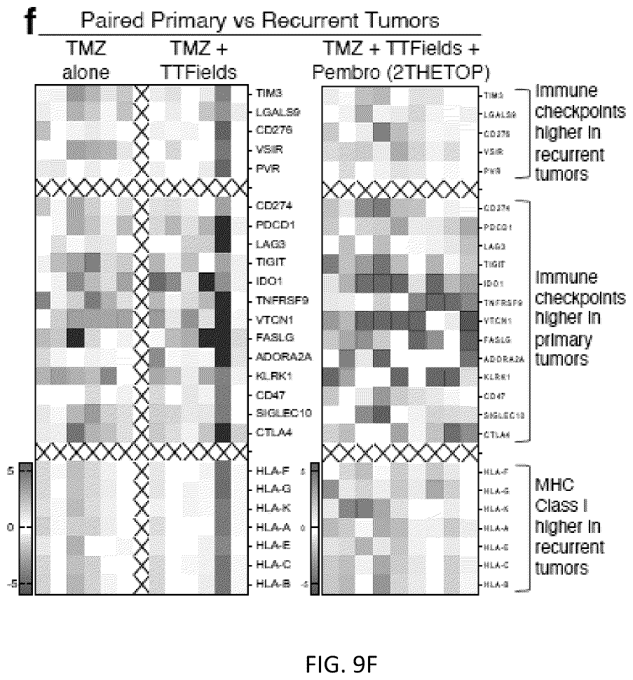

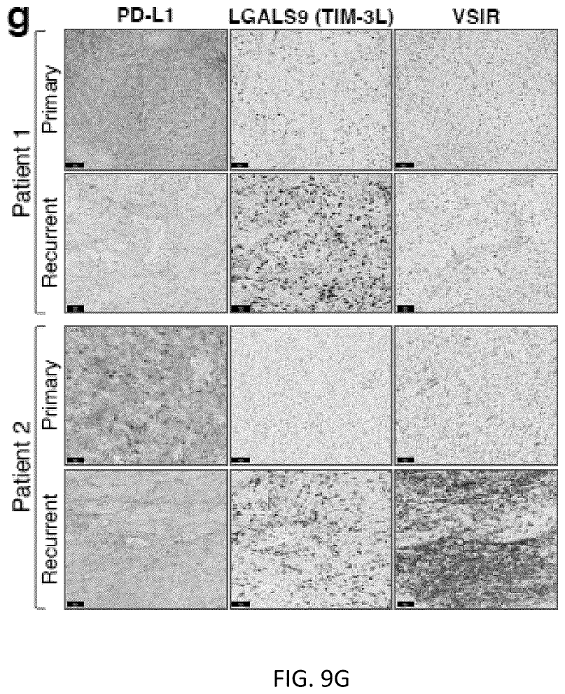

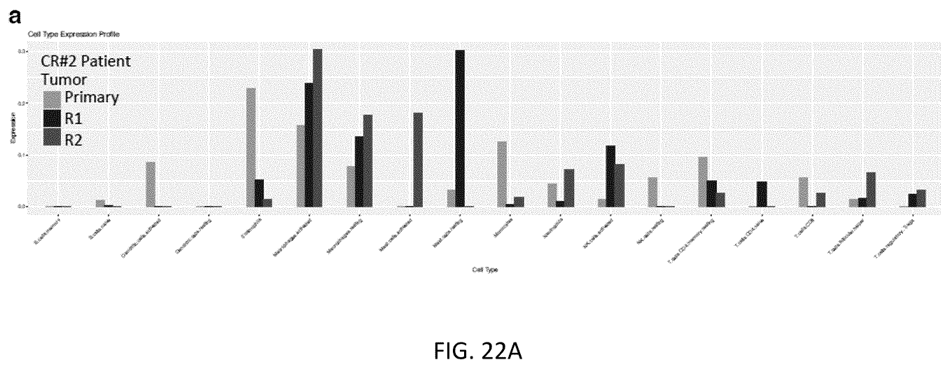

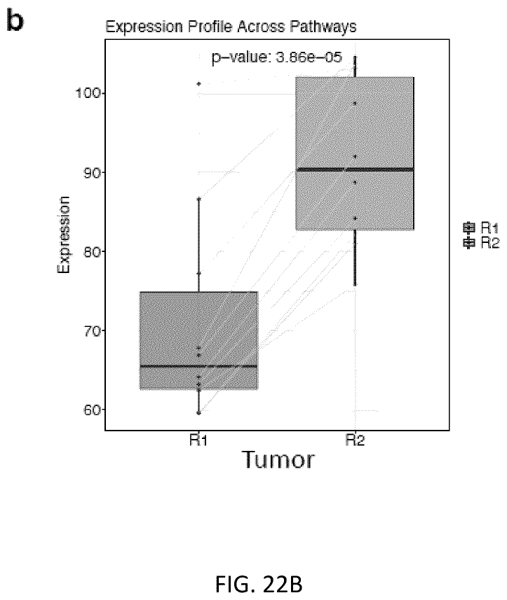

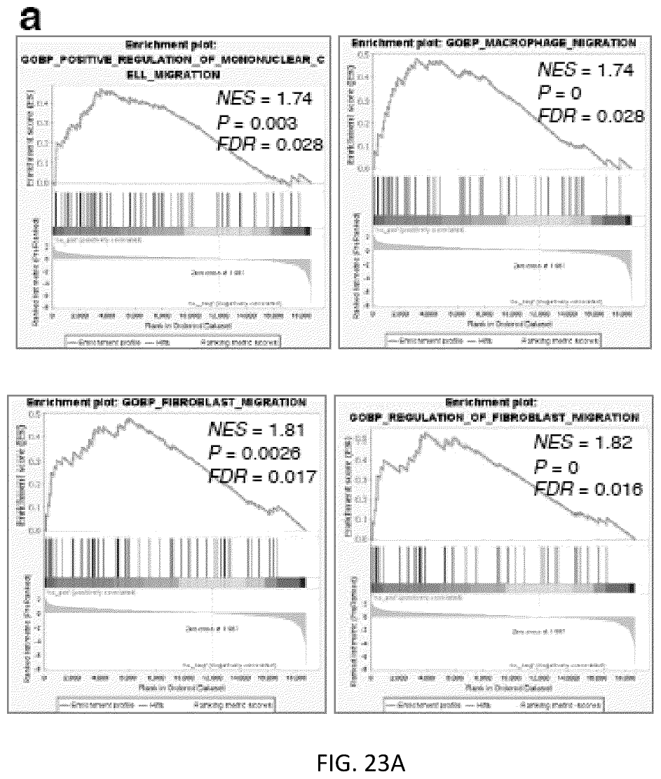

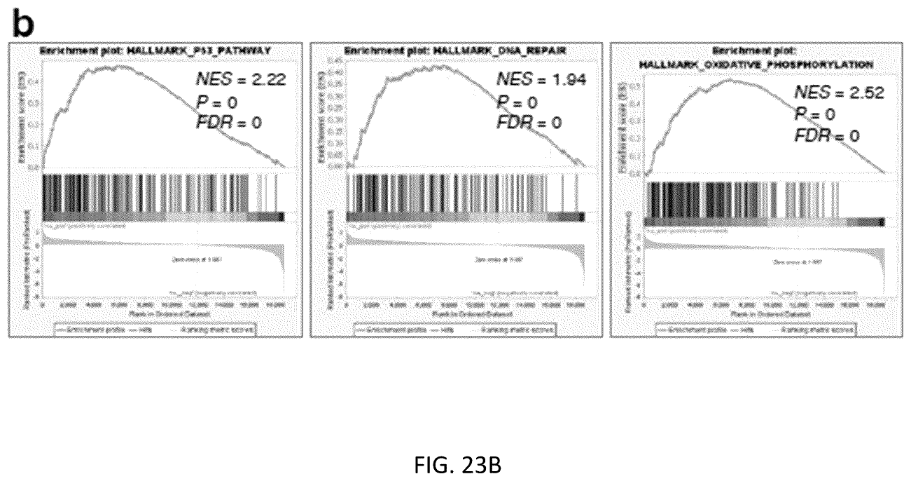

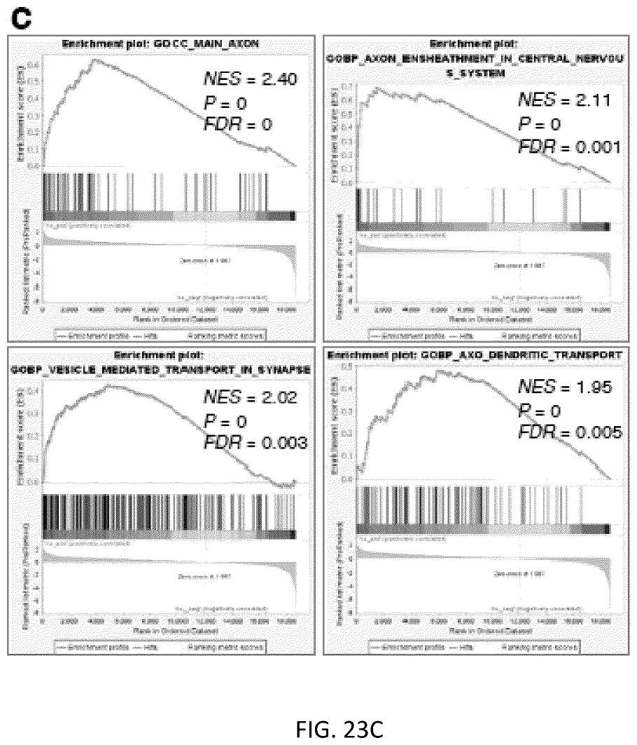

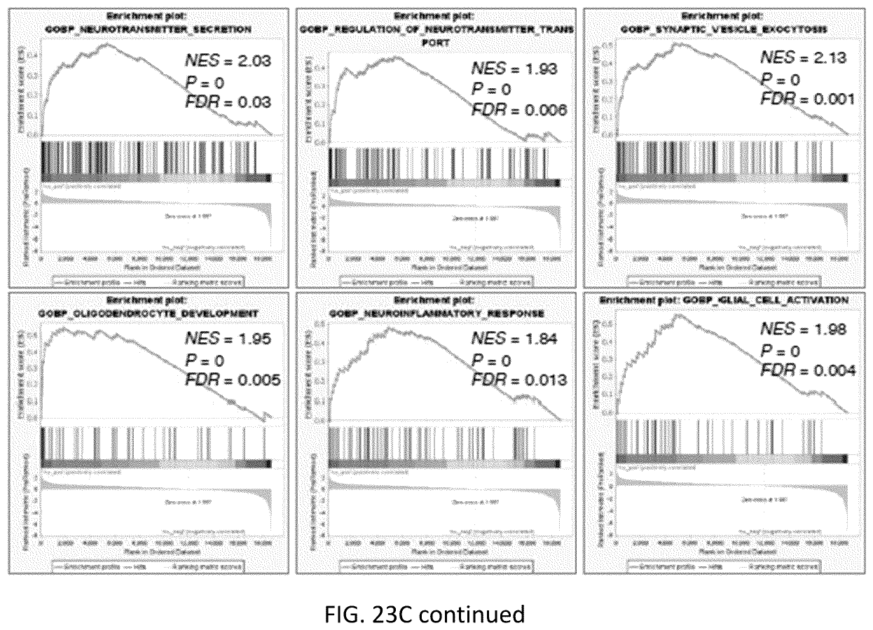

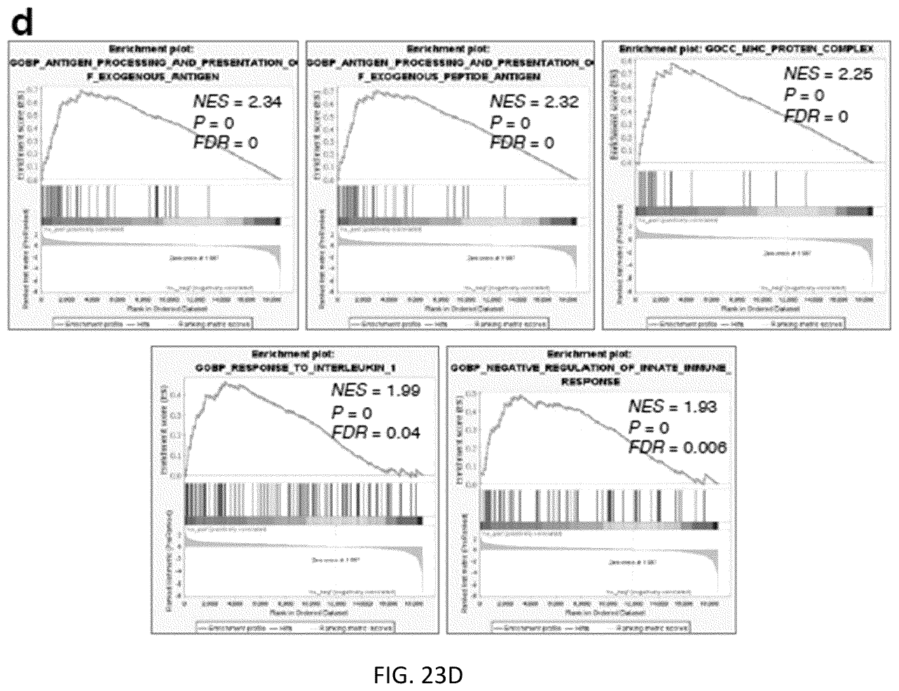

Concurrent with the systemic immune dysregulation observed in R2 blood, the TME of the R2 tumor also displayed heterogeneous immunological alterations. Immune cell deconvolution using bulk RNA-seq in the R2 tumor, relative to the R1 tumor, indicated a marked rise in myeloid lineage cells, such as macrophages/microglia, activated mast cells, monocytes, and neutrophils-all subtypes implicated in tumorigenesis (FIG. 22a). While CD4⁺ T cell counts were diminished in R2, CD8⁺ T cell presence increased compared to R1, paralleling intra-tumor TCR clonal expansion from R1 to R2 (FIG. 5b) and an upsurge in general and adaptive immune activation pathways (FIG. 22b), despite the overall clinical regression. This paradoxical state mirrors the dysfunctional peripheral T cell state previously described (FIG. 8d). To determine whether active TME reprogramming in recurrent tumors might undermine tumor-specific immune efficacy, global regulatory network shifts were examined in bulk RNA-seq profiles from 9 paired primary and recurrent tumor samples using GeneRep/nSCORE and revealed upregulation in recurrent tumors of regulatory hubs governing hypoxic response and fibrosis (hub 1.1), neural stemness (1.4), and notably immune response and inflammation (1.7) (FIG. 9a). Hub 1.1 included activated pathways in hypoxia and epithelial-mesenchymal transition (EMT), affecting the migration, proliferation, and genomic integrity of both tumor and stromal cells (FIG. 9b and FIG. 23a-b). These pathways are known to support GSC enrichment and augment TME-mediated immunosuppression. Both hubs 1.1 and 1.4 were involved in cell-cell interactions via focal adhesion and cell junction pathways, presumably through EMT by hub 1.1 and hub 1.4's role in neuronal properties including glial cell development and axonal and neurotransmitter signals (FIG. 9b and FIG. 23c), which GSCs may exploit to enhance glioma survival, invasiveness, and therapeutic resistance. Hub 1.7, reflecting the TME deconvolution findings, encompassed conflicting pathways—some fostering antigen-specific immune responses (FIG. 23d) and others enriched with genes facilitating pathological inflammation, recruitment of suppressive cells, and critically, the immune evasion tactics of GSCs (FIG. 9c).

To dissect the interplay between GBM cells and immune evasion mechanisms, the immune regulatory subnetworks inferred from deconvolved immune and non-immune cellular constituents (comprising tumor cells and other CD45⁻ stromal cells) were scrutinized in the TME of primary versus recurrent tumors. Compared to primary tumors, non-immune cells in recurrent GBM demonstrated reactivation of pathways modulated by the transcription factors CEBPB and ATF5. These transcription factors are pivotal in governing GSC properties, neuronal differentiation, metabolic processes, cellular migration, and immune evasion, particularly concerning inflammation and immune checkpoint pathways. For the immune compartment of the TME, the recurrent tumors' immune subnetwork was dominated by the senescence and metabolic regulator CREG1, which also steered a subnetwork within the non-immune TME cells impacting various immune checkpoint mechanisms. Notably, in the context of ongoing anti-PD-1 immunotherapy, downregulation of the PD-L1 axis in non-immune cells and PD-1 in immune cells were observed of recurrent tumors, along with IDO1, LAG3, and TIGIT checkpoints. In contrast, there was a significant upsurge in alternative immune checkpoints, specifically TIM-3 on immune cells and its ligand Galectin-9 (LGALS9), V-domain Ig suppressor of T cell activation (VISTA or VSIR), PVR (Poliovirus Receptor or CD155—a TIGIT ligand), and CD276 (B7-H3) on non-immune cells, suggesting a potential route of therapeutic resistance.

This pattern of adaptive resistance, characterized by elevated alternate immune checkpoints such as TIM-3 and LAG3, aligns with known resistance pathways in other solid tumors. Furthermore, the immune hub 1.7 also revealed several active pathways regulating alternative immune checkpoints post anti-PD-1 therapy, including PI3K/AKT/mTOR and TNFalpha/NFκB signaling. Specific overexpression of these alternate checkpoints were documented at both mRNA (FIG. 9f) and protein (representative targets-FIG. 9g) levels, in contrast to the downregulated PD-1/PD-L1 axis, TIGIT, and LAG3 in the nine paired tumor samples. Significant upregulation of MHC class I molecule expression was also noted in recurrent tumors, indicating that MHC I downregulation was not a predominant immune escape strategy in these cases. Additionally, the immune checkpoint alterations detected in the recurrent tumors of this study were not faithfully mirrored in an RNA-seq dataset from a historical cohort of GBM patients treated with either adjuvant TMZ alone or in combination with TTFields (FIG. 9f).

Collectively, these results demonstrate that adjuvant therapy combining TTFields, anti-PD-1 immunotherapy, and TMZ is safe and appears to confer survival benefits, especially for patients with large, inoperable tumors, who exhibited increased response rates, dynamic T cell clonal selection, and sustained adaptive immune responses, indicative of in situ vaccination from the study treatment. The concurrent downregulation of the PD-1/PD-L1 axis and the upregulation of alternative immune checkpoints might contribute to resistance mechanisms and tumor relapse.

### 3. Discussion

When pembrolizumab was added to the standard adjuvant therapy of TMZ and TTFields, patients generally tolerated it well. There were minimal severe immune-related adverse events (irAEs), and none of the patients had to discontinue treatment or died due to the therapy. Compared to the EF-14 study's TTFields plus TMZ group, the ITT GBM population in the 2THETOP study had markedly more negative prognostic indicators, such as a higher median age (60.5 vs. 56 years), a greater percentage of unmethylated MGMT promoters (73% vs. 54%), more cases of biopsy-only interventions (27% vs. 13%), a higher proportion of male participants (73% vs. 68%), and a lower KPS of 80% versus 90%. Moreover, the 2THETOP study reported a higher frequency of IDH1/2 mutations at 11.5%, compared to 7.3% in the EF-14 cohort; however, nearly half of the patients in the EF-14 study did not have tissue available for IDH status assessment, which was determined using IHC exclusively for the IDH1 R132H variant. In contrast, the 2THETOP study employed both IHC and next-generation sequencing to detect most variants in IDH1 and 2, reflecting a mutation rate consistent with the 12% IDH1 mutation rate observed in extensive genomic studies under the prior GBM classification. Although the non-comparative design of the single-arm study limits a definitive efficacy evaluation, the PFS and OS of the ITT GBM population in the 2THETOP study are noteworthy, especially in light of its unfavorable prognostic characteristics, exceeding historical survival data from the EF-14 study. However, a more accurate comparison would require a case-matched cohort from the EF-14 population. Nonetheless, the encouraging early survival results, along with the favorable safety profile, justify further research of this combination therapy in a randomized, placebo-controlled trial for patients with newly diagnosed GBM.

In preclinical GBM models, TTFields therapy has been demonstrated to serve as a complete tumor immunization platform by stimulating cGAS/STING and AIM2/Caspase-1 inflammasomes, thereby catalyzing a T1IFN-mediated immune initiation within the TME and periphery, in addition to triggering immunogenic tumor cell death. While it is challenging to directly observe these effects in the TME in patients with GBM due to the difficulty of repeated tissue sampling during treatment, the approach has been to characterize the indirect evidence indicative of TTFields' immunization impact. Remarkably, patients with biopsy-only GBM, who typically have a poorer prognosis, showed significantly improved PFS, OS, and response rates when treated with TTFields and pembrolizumab, compared to those who underwent maximal tumor resection and to the historical data showing the absence of efficacy associated with anti-PD-1 therapy without TTFields in newly diagnosed GBM, suggesting TTFields' in situ vaccination effect. Furthermore, the peripheral immune response was activated soon after TTFields treatment began, following a pattern dependent on the T1IFN pathway and T1IFN-specific immune cells. This response translated into T cell activation post TTFields application and intensified with subsequent pembrolizumab treatment, especially in patients with biopsy-only tumors. This implies that TTFields may reprogram the immune environment effectively, given the presence of bulky residual tumor. This phenomenon contrasts with the potential role of preceding chemoradiation-completed at least 4 weeks prior-which seems less likely to have an immediate impact on T cell activation, although a delayed effect cannot be categorically excluded. Moreover, TCR clonal replacement, indicating immune system engagement, occurred after starting TTFields and before anti-PD-1 immunotherapy. These changes, which intensified with pembrolizumab, were predictive of treatment response and particularly evident in patients with biopsy-only tumors. While ICIs are known to induce TCR clonal replacement by augmenting activated T cells or rejuvenating preexisting exhausted T cells, these findings suggest that preexisting expanded or exhausted T cell clones prior to TTFields therapy were likely supplanted by newly expanded clones due to TTFields, rather than contributing significantly to subsequent expansion by pembrolizumab. Indeed, while TCR clonal expansion and replacement occurred through different times in the first 3-4 months of treatment, only the TCR clonal replacement between C1 and C4 of pembrolizumab and not between Pre-TTF and C4 correlated with response and survival outcomes. Although TCR clonal replacement is hypothesized to occur in both CD4⁺ and CD8⁺ T cells in response to changes in antigen exposure or ICIs, most prior studies have focused on CD8⁺ T cells. However, the current study indicated that CD4⁺ T cells were the predominant subtype undergoing robust clonal replacement in response to TTFields and pembrolizumab, accentuating the adaptive helper role of CD4⁺ T cells in antitumor immunity, whereas CD8⁺ T cell clones were selected early and expanded at a steadier, more sustained rate. Whether this phenomenon is unique to TTFields and pembrolizumab is unclear and can be addressed in future studies comparing TTFields with TTFields plus pembrolizumab. Lastly, the influence of adjuvant TMZ on T1IFN-driven pathways, TCR clonal dynamics, and T cell activation was likely negligible, corroborated by the lack of any appreciable contribution by TMZ to the immune activating effects of TTFields in GBM models and the extensively documented immunosuppressive effects of TMZ, particularly at the standard dosing used in the 2THETOP study. Future research could focus on MGMT promoter-unmethylated GBM populations, where adjuvant TMZ might be excluded, to eliminate any potential confounding effects of TMZ on the immune phenotypes discussed herein.

Finally, in a meticulous tracking and evaluation of peripheral T cell clones throughout the course of treatment, a pronounced shift was observed in the T cell states of 2 patients who were classified as responders and had biopsy-only tumors. This shift was characterized by a progression from anergic and naive T cell states to those indicative of activated T cells, and ultimately, to central memory (CM) T cells. This dynamic transformation contrasted starkly with the relatively static T cell profiles of 2 non-responders who had undergone maximal tumor resection. Particularly noteworthy was the case of patient CR #2, who, after experiencing a sustained response for 24 months, demonstrated a reversal in T cell status. This “leftward shift” back towards an anergic and naive state, with a concurrent and significant reduction in CM T cells, was as striking as the initial shift towards activation. Intriguingly, this regression in T cell state did not manifest until the patient's second recurrence on the contralateral side. It is possible that certain interventions administered during the first recurrence-most notably dexamethasone—may have played a role in accelerating this shift. Coinciding with these changes in peripheral T cell status, the recurrent tumors in 9 primary and recurrent tumor pairs re-established an immunosuppressive TME reminiscent of the original primary tumors, albeit with a distinct composition of immune checkpoint mechanisms. This was evidenced by the heightened expression of several new checkpoint proteins, including TIM-3/LGALS9, VSIR, PVR, and CD276. While the upregulation of alternative immune checkpoints has been previously suggested as a mechanism of resistance to ICIs in various solid tumors, the analysis offers a granular view of the network changes within the TME. By comparing primary and recurrent tumors and employing deconvolution techniques, intricate alterations were delineated across both immune and non-immune TME constituents. This process appears to be governed by master regulatory elements that play established roles at the nexus of GSCs and immune evasion, particularly via immune checkpoints and cellular senescence pathways.

Overall, the insights garnered from this study underscore the potential benefits of integrating the in-situ vaccination effects of TTFields with anti-PD-1 immunotherapy. Furthermore, combining these treatments with additional ICIs targeting the newly identified checkpoints could potentially mitigate resistance and enhance overall treatment effectiveness.

### 4. Materials and Methods

i. Clinical Study Design

Disease assessment was performed using the modified Immunotherapy Response Assessment in Neuro-Oncology (iRANO) criteria. A complete response (CR) was defined as the disappearance of all enhancing measurable and non-measurable disease on a stable or decreasing steroid dose and sustained for at least 2 months. A partial response (PR) was defined as a ≥50% decrease in the sum of the products of perpendicular diameters of all measurable enhancing lesions on a stable or decreasing steroid dose sustained for at least 2 months.

ii. Bulk RNA-Seq of Enriched T Lymphocytes

Sample Processing: Using the human pan T Cell isolation kit (Miltenyi Biotec, Cat #130-096-535), untouched T cells were isolated from the PBMC single-cell suspensions in accordance with the manufacturer's guidelines. RNA extraction was performed using the QIAGEN RNeasy Midi Kit (Cat #75144), following the protocols provided by the manufacturer. Total tumor RNA was extracted from snap frozen and formalin-fixed paraffin-embedded tumor samples using QIAGEN RNeasy Midi Kit (Cat #75144) and RNeasy FFPE Kit (Cat #73504) separately. The bulk RNA-seq library construction, pooling, and sequencing were executed by NOVOGENE.

Pathway Differential Expression Analysis: Gene Set Enrichment Analysis (GSEA) was employed for the investigation of specific immune pathways of interest. In the context of comparing Maximal Resection versus Biopsy Only conditions, genes were ranked based on their logFoldChange derived from Gene differential expression analysis outcomes. Subsequently, GSEA in the preranked “classic” mode with 10,000 permutations was executed to ascertain the enrichment of desired pathways. The necessary command lines and the Java implementation for GSEA were acquired from the Broad Institute's software portal http://software.broadinstitute.org/gsea/index.jsp.

Boxplots for Pathway Activity: For each pathway among the top 10 identified via GSEA, boxplots were created to elucidate differences in pathway activity between samples from Maximal Resection and Biopsy Only groups across various timepoints. The calculation of pathway activity was based on the average expression values of genes constituting a pathway, with the relevant pathway gene sets being procured from the GSEA-MSigDB website http://www.gsea-msigdb.org.

Differential Expression Network/Cluster Analysis: The differential expression network analysis was performed using the GeneRep/nSCORE platform as previously described. The 3D GeneRep/nSCORE analysis plot was created by Blender (v 3.6). The gene interactions networks were generated using public availabile dataset. The 2 D gene network was visualized using Cytoscape software (v. 3.10.0).

iii. Single Cell RNA-Seq Analysis of PBMCs

Sample processing: All procedures involving human subjects complied with the ethical guidelines and approvals of the Institutional Review Board (IRB). Cryopreserved PBMCs obtained from patients were rinsed in PBS, and cell viability was assessed using Trypan Blue staining. Single-cell suspensions were then prepared and applied to the Chromium Single Cell Chip (10× Genomics) as per the instructions provided by the manufacturer. Subsequently, single-cell RNA-seq libraries were generated using the Chromium Next GEM Single Cell 5′ v2 (Dual Index). To ensure consistency, all patient samples and the corresponding libraries were processed simultaneously in a single batch. Sequencing of the single-cell libraries was performed on an Illumina NovaSeq system, utilizing an 8-base i7 sample index read, a 28-base read 1 for capturing cell barcodes and unique molecular identifiers (UMIs), and a 150-base read 2 for the mRNA insert.

Data Processing: The main operations were performed using the Seurat R package (3.2.2), unless otherwise stated. When option parameters for function deviated from the default values, details were provided of the changes accordingly. Most of the changes to the default options were made to accommodate and leverage the large size of the dataset. Cell Ranger Aggregation: The raw sequencing data was processed using cellranger mkfastq and cellranger multi commands of Cell Ranger package as described in TCR clonotyping section. Results from all libraries and batches were pooled together using the command cellranger aggr without normalization for dead cells as it will be handled downstream. The filtered background feature barcode matrix obtained from this step was used as input for sequential analysis. Normalization of UMI: Using the global scaling normalization method, the feature expression for each cell was divided by the total expression, multiplied by the scale factor (10,000), and log transformed using the Seurat R function NormalizeData with method “Log Normalize”. Seurat aggregation and correction for batch effect: As the counts were from three different batches, to align cells and eliminate batch effects for dimension reduction and clustering, the multi dataset integration strategy was adopted. Briefly, “anchors cells” were identified between pairs of datasets and used to normalize multiple datasets from different batches. A reference-based, reciprocal PCA variant of the method detailed in the Seurat R package was chosen. First, the previously integrated dataset was split by batches, using the Seurat function SplitObject. Next, for each split object, variable feature selection were performed using the function FindVariableFeatures. Features for integration were selected using the function SelectIntegrationFeatures and PCA performed for each split object on the selected features. The anchor cells were identified by using the function FindIntegrationAnchors with the reference chosen as the largest among 3 batches and the reduction option set to ‘rpca’. Finally, the whole datasets from 3 batches were reintegrated using the function IntegrateData with the identified anchor cells.

Uniform Manifold Approximation and Projection (UMAP) dimension reduction: The integrated multiple batch dataset was used as input for UMAP dimension reduction. The feature expression was scaled using the Seurat function ScaleData, followed by a PCA run using the function RunPCA (Seurat) with the total number of principal components (PC) to compute and store option of 100. The UMAP coordinates for single cells were obtained using the RunUMAP function (Seurat) with the top 75 PCs as input features (dims=1:75) with min.dist=0.75 and the number of training epochs n.epochs=2000. Clustering of cells: a graph-based clustering approach implemented in the Seurat package was relied on, which embeds cells in a K-nearest neighbor graph with edges drawn between similar cells and partitions nodes in the network into communities. Briefly, a Shared Nearest Neighbor graph was constructed using the FindNeigbhors function with an option dimension of reduction input dims=1:75, error bound nn.eps=0.5. This function calculates the neighborhood overlap (Jaccard index) between every cell and its k.param nearest neighbors. The graph was partitioned into clusters using the FindClusters function with different values for resolution parameter. The differential expressed gene markers for each cluster were found using the FindAllMarkers function with the option of only returning positive markers and a minimal fraction of cells with the marker of 0.25. The default Wilcoxon Rank Sum test was used to calculate statistical differences in each cell cluster.

Cell Type Annotation: To delineate specific cell types within the data, cell type labels were assigned manually to clusters emerging from UMAP analysis. This annotation process was guided by the expression profiles of a set of marker genes, which are characteristic of various cell types including T Cells, B Cells, Natural Killer (NK) cells, Monocytes, Dendritic Cells (DC), Myeloid-Derived Suppressor Cells (MDSC), Megakaryocytes, Red Blood Cells (RBC), CD34+ stem cells, Granulocytes, Lymphocytes, Macrophages, Basophils, Eosinophils, and Neutrophils. The marker genes utilized for this purpose encompassed a wide array of immune response and cell differentiation indicators such as CD3D, CD3E, ID3, IL7R, CCR7, ITGB1, CD95, TCF7, CD3D, CD3E, CD4, S100A4, CCR10, FOXP3, IL2RA, TNFRSF18, IKZF2, CTLA4, IL2, IL4, IL13, IL17A, CD3D, CD3E, CD8A, CD8B, CCL4, GZMA, GZMB, GZMH, GZMM, GZMK, IFNG, GNLY, TNF, PDCD1, LAG3, CD79A, CD79B, CD19, JCHAIN, GNLY, NKG7, CD16, NCAM1, KIR2DL4, SIGLEC7, CD14, LYZ, S100A8, S100A9, LGALS3, FCN1, FCGR3A, MS4A7, FCER1A, CST3, ITGAM, ITGAX, CLEC10A, CLEC9A, THBD, CD1C, LILRA4, CLEC4C, TLR7, TLR9, ITGAM, CD33, CD3D, CD3E, CD14, CD19, FUT4, CEACAM1, HLA-DRA, HLA-DRB1, HLA-DRB5, PPBP, PF4, ITGA2B, ITGB3, PEAR1, CD42D, CD59, HBG1, HBG2, HBB, CD34, CCR3, CD11b, CD13, CD18, CD229, CRACC, CD14, CD68, CD36, CD164, LAMP1, CD44, CD69, EMR1, MPO, CD62L, CD3D, CD3E, CD4, CD8A, CD8B, NKG7, GNLY, CD14, LYZ, FCER1A, CLEC10A, LILRA4, CLEC4C, CD79A, CD79B, HBB, PPBP, PF4. T cells were further divided into clusters to annotate subpopulations: Naive CD4, Central Memory CD4, Central Memory CD8, Anergic CD4, Activated CD4, Treg, Exhausted CD4, Stem-like CD8, NKT, Exhausted CD8, Effector CD8, Naive CD8, Cytotoxic CD4 and Effector Memory CD8 using the following marker genes: CD3D, CD4, CD8A, CTLA4, PDCD1, TIGIT, FOXP3, CCR7, GZMK, GZMB, GZMH, IL7R, CCL5, KLRB1, TRAV16, TRAV17, CX3CR1, CCL4, TRDC, CD69, FOS.

UMAP showing Pathway Activity: T cells were focused on. The objective was to examine the pathway activity within these T cells across various patient timepoints. This involved calculating the mean expression levels of genes associated with each pathway, a method analogous to that used in bulk RNA sequencing data analysis. The mean expression levels were then normalized against the baseline time (Pre-TTF), facilitating the observation of dynamic changes in pathway activity. The UMAP visualizations were generated using the FeaturePlot function in the Seurat package.

Violin Plot showing Pathway Activity: Pathway activity was quantified using the same methodology as described for the UMAP analysis. This approach also incorporated additional data points, specifically the number of cells present at each timepoint and the statistical significance (p-value) of expression changes between timepoints compared to the Pre-TTF baseline. The significance levels were determined using the FindMarker function of the Seurat package, which assesses differential expression.

iv. TCR Clonotyping

Sample Processing: The Human V(D)J Amplification Kit (10× Genomics) Single-cell RNA-seq libraries were generated using the Chromium Next GEM Single Cell 5′ v2 (Dual Index) alongside the Human V(D)J Amplification Kit (10× Genomics), following the manufacturer's protocols. To ensure consistency, all patient samples and the corresponding libraries were processed simultaneously in a single batch. Sequencing of the single-cell libraries was performed on an Illumina NovaSeq system, utilizing an 8-base i7 sample index read, a 28-base read 1 for capturing cell barcodes and unique molecular identifiers (UMIs), and a 150-base read 2 for the mRNA insert.

Data Processing: The 5′ Single Cell V(D)J library data was first processed using 10× Genomics Cell Ranger package (v. 7.0.0, with java/9.0.1, bcl2fastq/2.20.0.422 dependencies). Command cellranger mkfastq was used to convert the raw sequencing data from the bel to fastq format, and cellranger multi command to align to the reference genomes GRCh38 (GENCODE v.24) and single cell clonal identification. Clonal tracking plots were created using the Immunoarch R package v0.9.0 (https://cloud.r-project.org/web/packages/immunarch/index.html) with the function trackClonotypes, option col=“a.a”, to collapse all clones that share the same amino acid sequences. TCR clones of immune cells from bulk primary and recurrent tumor samples were analyzed by ImmunoSeq proprietary pipeline of Adaptive Biotech. A clonal tracking grid was generated for CD8 and CD4 T Cells. In the grid, all TCR clones were identified at a particular timepoint and then tracked (number of cells, cell type change, pathway activity, etc.) for each timepoint. For example, the first row tracks all TCR clones at timepoint Pre-TTF, second row tracks all TCR clones at the next timepoint, and so on. The same was done for top 2 clones instead of all clones for an in-depth look.

TCR Clonotyping, Clonal Evolution, and Activation: A key part of this analysis involved tracking the evolution and activation of T Cell Receptor (TCR) clones over time. A clonal tracking grid was established to map the presence and characteristics of TCR clones at each patient timepoint, focusing on aspects such as the number of cells, changes in cell type, and pathway activity. For example, the first row in the grid tracks all TCR clones at timepoint Pre-TTF, second row tracks all TCR clones at the next patient timepoint, and so on. This tracking was performed for all identified clones, with a detailed analysis for the top two clones, offering insights into the dynamic nature of T cell responses.

TCR Clonal replacement ratio calculation: The TCR clonal replacement ratio was calculated between two time points t1 and t2 was calculated as followed. The top clones at time point 1 was tracked in the time point 2 and their proportion in t2 was recorded (t1_top clone proportion at t2). Also, the proportion of the top clones at time 2 was calculated (t2_top clone proportion at t2).

Clonal replacement=t2_top clone proportion at t2/max (t1_top clone proportion at t2, 0.001).

The small number 0.001 was added to prevent division to zero. The p-value for clonal replacement was calculated using Student's T-test, with null hypothesis that the clonal replacement ratio equal 1 and the alternative hypothesis is the clonal replacement ratio is greater than 1. To assess the role of TCR clonal replacement in patient survival, the Cox Proportional Hazards Model was created, using coxph and survfit commands in R survival package (v3.5.7) with co-variates: Age, Sex, MGMT.methylation, IDH.1.mutation (for ITT GBM cohort), and TCR clonal replacement ratio. The Kaplan Meyer plot was calculated using median replacement ratio to divide patients in two groups with low and high replacement ratio by survfit, survdiff (R survival package) and plotted using autoplot function of ggplot2 package (3.4.4). The p-value is calculated using log rank test.

v. Immunohistochemistry

IHC was performed in the USC Immunohistochemistry R&D Laboratory using 5-μm sections of formalin-fixed paraffin-embedded specimens. Slides were run on a Leica Bond III Autostainer. EDTA (High pH 9.0, for LGALS9 and PD-L1) and citrate (low pH 6.0, for VSIR), were used to retrieval antigen. The slides were then incubated with correlated antibodies for 15 minutes followed by BOND IHC Polymer Detection Kit (Leica, Cat #DS9800): anti-LGALS9 (Sigma-Aldrich, Cat #MABT833, 1:850 dilution), anti-PD-L1 (Abcam, Cat #AB205921, 1:100 dilution), and anti-VSIR (Abcam, Cat #AB300042. 1:100 dilution). The stains were counterstained with hematoxylin and allowed to dry before they were scanned at 40× with the Phillips FMT0095.

RNA-seq data deposit: Accession number in the Gene Expression Omnibus (GEO).

vi. Study Approval

Human subject work was performed accordingly to approved protocol from the IRB at the University of Florida and University of Southern California. An informed consent was obtained from each human participant before study procedure and analysis were performed.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having a biopsy-only glioblastoma tumor comprising:
applying an alternating electric field to a target site of the subject for a period of time, wherein the target site comprises one or more glioblastoma cells;
administering a therapeutically effective amount of temozolomide (TMZ); and
administering a therapeutically effective amount of a checkpoint inhibitor to the subject.

2. The method of claim 1, wherein the checkpoint inhibitor is pembrolizumab (KEYTRUDA®), ipilimumab (YERVOY®), nivolumab (OPDIVO®), cemiplimab (trade name LIBTAYO®), and dostarlimab (JEMPERLI), atezolizumab (TECENTRIQ®), durvalumab (IMFINZI®), or avelumab (BAVENCIO®).

3. The method of claim 2, wherein the checkpoint inhibitor is pembrolizumab.

4. The method of claim 1, wherein the subject has previously undergone standard of care TMZ treatment and/or radiation therapy.

5. The method of claim 1, wherein antigen-specific T cell stimulation is increased in the subject.

6. The method of claim 1, wherein T cell receptor (TCR) clonal turnover is increased in the subject.

7. The method of claim 1, wherein central memory T cell development is increased in the subject.

8. The method of claim 7, wherein the increase of antigen-specific T cell stimulation and/or T cell receptor (TCR) clonal turnover and/or central memory T cell development is higher in a biopsy-only subject compared to a subject having maximal tumor resection.

9. The method of claim 1, wherein subject with biopsy-only tumors has improved progression-free survival, overall survival, and response rates compared to a subject who underwent maximal tumor resection.

10. The method of claim 1, wherein CD4+ T cells are the predominant T cell subtype undergoing robust clonal replacement.

11. A method of increasing survival of a subject having a biopsy-only glioblastoma tumor comprising:
applying an alternating electric field to a target site of the subject for a period of time, wherein the target site comprises one or more glioblastoma cells;
administering a therapeutically effective amount of temozolomide (TMZ); and
administering a therapeutically effective amount of a checkpoint inhibitor to the subject.

12. The method of claim 11, wherein the checkpoint inhibitor is Pembrolizumab (Keytruda), ipilimumab (Yervoy), nivolumab (Opdivo), cemiplimab (trade name Libtayo), and dostarlimab (Jemperli), atezolizumab (Tecentriq), durvalumab (imfinzi), or avelumab (Bavencio).

13. The method of claim 12, wherein the checkpoint inhibitor is pembrolizumab.

14. The method of claim 11, wherein the subject has previously undergone standard of care TMZ treatment and/or radiation therapy.

15. The method of claim 11, wherein antigen-specific T cell stimulation is increased in the subject.

16. The method of claim 11, wherein T cell receptor (TCR) clonal turnover is increased in the subject.

17. The method of claim 11, wherein central memory T cell development is increased in the subject.

18. The method of claim 17, wherein the increase of antigen-specific T cell stimulation and/or T cell receptor (TCR) clonal turnover and/or central memory T cell development is higher in a biopsy-only subject compared to a subject having maximal tumor resection.

19. The method of claim 11, wherein subject with biopsy-only tumors has improved progression-free survival, overall survival, and response rates compared to a subject who underwent maximal tumor resection.

20. The method of claim 11, wherein CD4+ T cells are the predominant T cell subtype undergoing robust clonal replacement.
